(19) United States
(12) Patent Application Publication
(10) Pub. No.: US 2010/0144715 A1
(43) Pub. Date: Jun. 10, 2010

(54) Substituted Benzodiazepinones, Benzoxazepinones and Benzothiazepinones as Sodium Channel Blockers

(76) Inventors: Scott B. Hoyt, Hoboken, NJ (US); Dong Ok, Colonia, NJ (US); Hyun Ok, Colonia, NJ (US); Clare London, Chatham, NJ (US); Joseph I. Duffy, Cranford, NJ (US)

Correspondence Address: MERCK, P O BOX 2000, RAHWAY, NJ, 07065-0907, US

(21) Appl. No.: 12/528,041

(22) PCT Filed: Feb. 25, 2008
(86) PCT No.: PCT/US08/02441

§ 371 Date: Dec. 1, 2009

Related U.S. Application Data
(60) Provisional application No. 60/904,065, filed on Feb. 28, 2007.

(51) Int. Cl. A61K 31/551; C07D 243/12; C07D 281/08; A61K 31/553; A61K 31/554; A61P 25/08

(52) U.S. Cl. 514/221; 540/517; 540/491; 514/224.2; 514/230.5

(57) ABSTRACT

The present invention is directed to substituted benzodiazepinones, benzoxazepinones and benzothiazepinones compounds that are sodium channel blockers useful for the treatment of chronic and neuropathic pain. The compounds of the present invention are also useful for the treatment of other conditions, including disorders of the CNS such as anxiety, depression, epilepsy, manic depression and bipolar disorder. This invention also provides pharmaceutical compositions comprising a compound of the present invention, either alone, or in combination with one or more therapeutically active compounds, and a pharmaceutically acceptable carrier. This invention further comprises methods for the treatment of acute pain, chronic pain, visceral pain, inflammatory pain, neuropathic pain and disorders of the CNS including, but not limited to, epilepsy, manic depression, depression, anxiety and bipolar disorder comprising administering the compounds and pharmaceutical compositions of the present invention.

### FIELD OF THE INVENTION

The present invention is directed to a series of benzodiazepinones, benzoxazepinones and benzothiazepinones compounds that are sodium channel blockers useful for the treatment of chronic and neuropathic pain. The compounds of the present invention are also useful for the treatment of other conditions, including disorders of the nervous system such as postherpetic neuralgia, diabetic neuropathy, epilepsy, manic depression, bipolar disorder, depression, anxiety and urinary incontinence.

### BACKGROUND OF THE INVENTION

Voltage-gated ion channels allow electrically excitable cells to generate and propagate action potentials and therefore are crucial for nerve and muscle function. Sodium channels play a special role by mediating rapid depolarization, which constitutes the rising phase of the action potential and in turn activates voltage-gated calcium and potassium channels. Voltage-gated sodium channels represent a multigene family. Nine sodium channel subtypes have been cloned and functionally expressed to date. [Clare, J. J., Tate, S. N., Nobbs, M. & Romanos, M. A. Voltage-gated sodium channels as therapeutic targets. Drug Discovery Today 5, 506-520 (2000)]. They are differentially expressed throughout muscle and nerve tissues and show distinct biophysical properties. All voltage-gated sodium channels are characterized by a high degree of selectivity for sodium over other ions and by their voltage-dependent gating. [Catterall, W. A. Structure and function of voltage-gated sodium and calcium channels. Current Opinion in Neurobiology 1, 5-13 (1991)]. At negative or hyperpolarized membrane potentials, sodium channels are closed. Following membrane depolarization, sodium channels open rapidly and then inactivate. Sodium channels only conduct currents in the open state and, once inactivated, have to return to the resting state, favored by membrane hyperpolarization, before they can reopen. Different sodium channel subtypes vary in the voltage range over which they activate and inactivate as well as in their activation and inactivation kinetics.

Sodium channels are the target of a diverse array of pharmacological agents, including neurotoxins, antiarrhythmics, anticonvulsants and local anesthetics. [Clare, J. J., Tate, S. N., Nobbs, M. & Romanos, M. A. Voltage-gated sodium channels as therapeutic targets. Drug Discovery Today 5, 506-520 (2000)]. Several regions in the sodium channel secondary structure are involved in interactions with these blockers and most are highly conserved. Indeed, most sodium channel blockers known to date interact with similar potency with all channel subtypes. Nevertheless, it has been possible to produce sodium channel blockers with therapeutic selectivity and a sufficient therapeutic window for the treatment of epilepsy (e.g. lamotrigine, phenyloin and carbamazepine) and certain cardiac arrhythmias (e.g. lignocaine, tocamide and mexiletine).

It is well known that the voltage-gated Na⁺ channels in nerves play a critical role in neuropathic pain. Injuries of the peripheral nervous system often result in neuropathic pain persisting long after the initial injury resolves. Examples of neuropathic pain include, but are not limited to, postherpetic neuralgia, trigeminal neuralgia, diabetic neuropathy, chronic lower back pain, phantom limb pain, pain resulting from cancer and chemotherapy, chronic pelvic pain, complex regional pain syndrome and related neuralgias. It has been shown in human patients as well as in animal models of neuropathic pain, that damage to primary afferent sensory neurons can lead to neuroma formation and spontaneous activity, as well as evoked activity in response to normally innocuous stimuli. [Carter, G. T. and B. S. Galer, Advances in the management of neuropathic pain. Physical Medicine and Rehabilitation Clinics of North America, 2001. 12(2): p. 447-459]. The ectopic activity of normally silent sensory neurons is thought to contribute to the generation and maintenance of neuropathic pain. Neuropathic pain is generally assumed to be associated with an increase in sodium channel activity in the injured nerve. [Baker, M. D. and J. N. Wood, Involvement of Na channels in pain pathways. TRENDS in Pharmacological Sciences, 2001. 22(1): p. 27-31].

Indeed, in rat models of peripheral nerve injury, ectopic activity in the injured nerve corresponds to the behavioral signs of pain. In these models, intravenous application of the sodium channel blocker and local anesthetic lidocaine can suppress the ectopic activity and reverse the tactile allodynia at concentrations that do not affect general behavior and motor function. [Mao, J. and L. L. Chen, Systemic lidocaine for neuropathic pain relief. Pain, 2000. 87: p. 7-17]. These effective concentrations were similar to concentrations shown to be clinically efficacious in humans. [Tanelian, D. L. and W. G. Brose, Neuropathic pain can be relieved by drugs that are use-dependent sodium channel blockers: lidocaine, carbamazepine and mexiletine. Anesthesiology, 1991. 74(5): p. 949-951]. In a placebo-controlled study, continuous infusion of lidocaine caused reduced pain scores in patients with peripheral nerve injury, and in a separate study, intravenous lidocaine reduced pain intensity associated with postherpetic neuralgia (PHN). [Mao, J. and L. L. Chen, Systemic lidocaine for neuropathic pain relief. Pain, 2000. 87: p. 7-17. Anger, T., et al., Medicinal chemistry of neuronal voltage-gated sodium channel blockers. Journal of Medicinal Chemistry, 2001. 44(2): p. 115-137]. Lidoderm®, lidocaine applied in the form of a dermal patch, is currently the only FDA approved treatment for PHN. [Devers, A. and B. S. Galer, Topical lidocaine patch relieves a variety of neuropathic pain conditions: an open-label study. Clinical Journal of Pain, 2000. 16(3): p. 205-208].

In addition to neuropathic pain, sodium channel blockers have clinical uses in the treatment of epilepsy and cardiac arrhythmias. Recent evidence from animal models suggests that sodium channel blockers may also be useful for neuroprotection under ischaemic conditions caused by stroke or neural trauma and in patients with multiple sclerosis (MS). [Clare, J. J., et al. And Anger, T., et al.].

### SUMMARY OF THE INVENTION

The present invention is directed to substituted benzodiazepinones, benzoxazepinones and benzothiazepinones compounds that are sodium channel blockers useful for the treatment of chronic and neuropathic pain. The compounds of the present invention are also useful for the treatment of other conditions, including disorders of the CNS such as anxiety, depression, epilepsy, manic depression and bipolar disorder. This invention also provides pharmaceutical compositions comprising a compound of the present invention, either alone, or in combination with one or more therapeutically active compounds, and a pharmaceutically acceptable carrier.

This invention further comprises methods for the treatment of acute pain, chronic pain, visceral pain, inflammatory pain, neuropathic pain and disorders of the CNS including, but not limited to, epilepsy, manic depression, depression, anxiety and bipolar disorder comprising administering the compounds and pharmaceutical compositions of the present invention.

### DETAILED DESCRIPTION OF THE INVENTION

The present invention comprises compounds represented by formula (I):

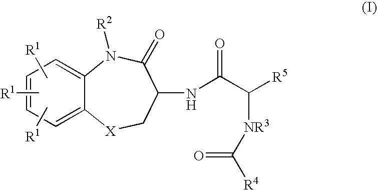

and pharmaceutically acceptable salts thereof, wherein
each R¹ is independently selected from the group consisting of
hydrogen,
halogen,
cyano,
C₁₋₆ alkyl, unsubstituted or substituted with one to five halogens, and
C₁₋₆ alkoxy, unsubstituted or substituted with one to five halogens;
   R² is independently selected from the group consisting of
hydrogen,
C₁₋₆ alkyl, unsubstituted or substituted with one to six substituents selected from halogen and hydroxy,
C₁₋₆ alkenyl,
C₁₋₆ alkynyl,
C₁₋₆ alkoxy-C₁₋₆ alkylene, unsubstituted or substituted with one to six halogens,
C₁₋₆ cycloalkyl, wherein cycloalkyl is unsubstituted or substituted with one to six substituents independently selected from halogen, cyano, C₁₋₆ alkyl and C₁₋₆ alkoxy, wherein alkyl and alkoxyl are unsubstituted or substituted with one to six halogens, and
C₁₋₆ cycloalkyl-C₁₋₆alkylene, wherein cycloalkyl is unsubstituted or substituted with one to six substituents independently selected from halogen, cyano, C₁₋₆ alkyl and C₁₋₆ alkoxy, wherein alkyl and alkoxyl are unsubstituted or substituted with one to six halogens;
   R³ is independently selected from the group consisting of
hydrogen and
C₁₋₆ alkyl;
   R⁴ is independently selected from the group consisting of
C₁₋₁₀ alkyl, unsubstituted or substituted with one to six halogens,
C₁₋₁₀ alkoxy, unsubstituted or substituted with one to six halogens,
C₁₋₁₀ cycloalkyl-C₀₋₆ alkylene, wherein cycloalkyl is unsubstituted or substituted with one to six substituents independently selected from halogen, cyano, C₁₋₆ alkyl and C₁₋₆ alkoxy, wherein alkyl and alkoxyl are unsubstituted or substituted with one to six halogens,
—(CH₂)ₘ-aryl wherein m is 0, 1, 2 or 3, and wherein aryl is unsubstituted or substituted with one to five substituents independently selected from halogen, cyano, C₁₋₆ alkyl and C₁₋₆ alkoxy, wherein alkyl and alkoxy are unsubstituted or substituted with one to six halogens, and
—(CH₂)ₘ-heteroaryl wherein m is 0, 1, 2 or 3, and wherein heteroaryl is unsubstituted or substituted with one to five substituents independently selected from halogen, C₁₋₆ alkyl and C₁₋₆ alkoxy, wherein alkyl and alkoxy are unsubstituted or substituted with one to six halogens;
   R⁵ is independently selected from the group consisting of
—(CH₂)ₙ-aryl wherein n is 0, 1, or 2, and wherein aryl is unsubstituted or substituted with one to five substituents independently selected from hydroxy, halogen, cyano, C₁₋₆ alkyl and C₁₋₆ alkoxy, wherein alkyl and alkoxy are unsubstituted or substituted with one to six halogens,
—(CH₂)ₙ-heteroaryl wherein n is 0, 1 or 2, and wherein aryl is unsubstituted or substituted with one to five substituents independently selected from halogen,
C₁₋₆ alkyl and C₁₋₆ alkoxy, wherein alkyl and alkoxy are unsubstituted or substituted with one to six halogens;
   X is independently selected from the group consisting of
oxygen,
nitrogen, unsubstituted or substituted with one R⁶ as defined herein,
sulfur,
sulfoxide, and
sulfone;
   R⁶ is independently selected from the group consisting of
C₁₋₁₀ alkyl, unsubstituted or substituted with one to six halogens,
C₁₋₁₀ alkoxy, unsubstituted or substituted with one to six halogens,
C₁₋₁₀ cycloalkyl-C₀₋₆ alkylene, wherein cycloalkyl is unsubstituted or substituted with one to six substituents independently selected from halogen, cyano, C₁₋₆ alkyl and C₁₋₆ alkoxy, wherein alkyl and alkoxyl are unsubstituted or substituted with one to six halogens,
—(CH₂)ₚ-aryl wherein p is 0, 1, 2 or 3, and wherein aryl is unsubstituted or substituted with one to five substituents independently selected from halogen, cyano, C₁₋₆ alkyl and C₁₋₆ alkoxy, wherein alkyl and alkoxy are unsubstituted or substituted with one to six halogens, and
—(CH₂)ₚ-heteroaryl wherein p is 0, 1, 2 or 3, and wherein heteroaryl is unsubstituted or substituted with one to five substituents independently selected from halogen, C₁₋₆ alkyl and C₁₋₆ alkoxy, wherein alkyl and alkoxy are unsubstituted or substituted with one to six halogens.

In one embodiment of the compounds of the present invention, X═N as depicted in formula (Ia)

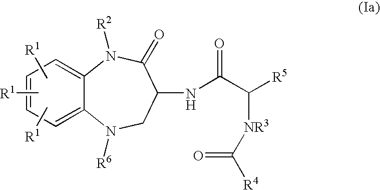

and pharmaceutically acceptable salts thereof, wherein R¹, R², R³, R⁴, R⁵ and R⁶ are as defined herein.

In a class of this embodiment of the compounds of the present invention, the carbon atoms marked with * and ** have the stereochemical configurations depicted in formula (Ib)

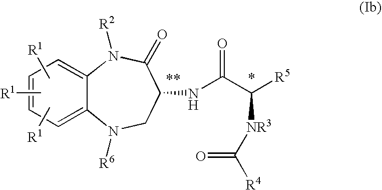

and pharmaceutically acceptable salts thereof, wherein R¹, R², R³, R⁴, R⁵ and R⁶ are as defined herein.

In a subclass of this embodiment of the compounds of the present invention, each R¹═H as depicted in formula (Ic)

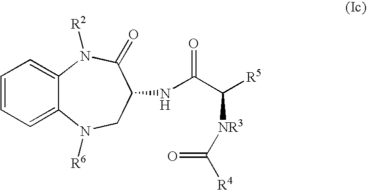

and pharmaceutically acceptable salts thereof, wherein R², R³, R⁴, R⁵ and R⁶ are as defined herein.

Within this subclass the invention encompasses compounds for formula (Ic) wherein:

R² is independently selected from the group consisting of
hydrogen,
C₁₋₆ alkyl, unsubstituted or substituted with one to six substituents selected from halogen and hydroxy,
C₁₋₆ alkenyl, and
C₁₋₆ alkoxy-C₁₋₆ alkylene, unsubstituted or substituted with one to six halogens;
   R³ is hydrogen;
   R⁴ is independently selected from the group consisting of
C₁₋₆ alkyl, unsubstituted or substituted with one to six halogens,
C₁₋₆ alkoxy, unsubstituted or substituted with one to six halogens,
C₃₋₆ cycloalkyl-C₀₋₆ alkylene, wherein cycloalkyl is unsubstituted or substituted with one to six substituents independently selected from halogen, cyano,
C₁₋₆ alkyl and C₁₋₆ alkoxy, wherein alkyl and alkoxy are unsubstituted or substituted with one to six halogens, and
phenyl, wherein phenyl is unsubstituted or substituted with one to five substituents independently selected from halogen, cyano, C₁₋₆ alkyl and C₁₋₆ alkoxy, wherein alkyl and alkoxy are unsubstituted or substituted with one to six halogens,

### R⁵ is

—CH₂-phenyl, wherein phenyl is unsubstituted or substituted with one to five substituents independently selected from hydroxy, halogen, cyano, C₁₋₆ alkyl and C₁₋₆ alkoxy, wherein alkyl and alkoxy are unsubstituted or substituted with one to six halogens; and
   R⁶ is independently selected from the group consisting of
C₁₋₁₀ alkyl, unsubstituted or substituted with one to six halogens,
C₁₋₁₀ cycloalkyl-C₀₋₆ alkylene, wherein cycloalkyl is unsubstituted or substituted with one to six substituents independently selected from halogen, cyano, C₁₋₆ alkyl and C₁₋₆ alkoxy, wherein alkyl and alkoxyl are unsubstituted or substituted with one to six halogens, and
—(CH₂)ₚ-phenyl wherein p is 0, 1, 2 or 3, and wherein phenyl is unsubstituted or substituted with one to five substituents independently selected from halogen, cyano, C₁₋₆ alkyl and C₁₋₆ alkoxy, wherein alkyl and alkoxy are unsubstituted or substituted with one to six halogens.

In a second embodiment of the compounds of the present invention, X═O as depicted in formula (Id)

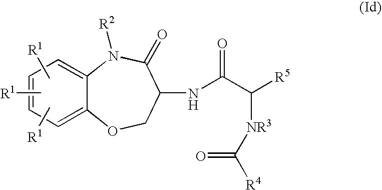

and pharmaceutically acceptable salts thereof, wherein R¹, R², R³, R⁴ and R⁵ are as defined herein.

In a class of this embodiment of the compounds of the present invention, the carbon atom marked with a * has the stereochemical configuration depicted in formula (Ie)

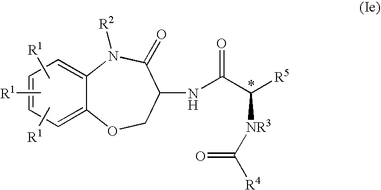

and pharmaceutically acceptable salts thereof, wherein R¹, R², R³, R⁴ and R⁵ are as defined herein.

Within this class, the invention encompasses compounds of Formula (Ie) wherein:

R² is independently selected from the group consisting of
hydrogen,
C₁₋₆ alkyl, unsubstituted or substituted with one to six substituents selected from halogen and hydroxy,
C₁₋₆ alkenyl, and
C₁₋₆ alkoxy-C₁₋₆ alkylene, unsubstituted or substituted with one to six halogens;
   R³ is hydrogen;
   R⁴ is independently selected from the group consisting of
C₁₋₆ alkyl, unsubstituted or substituted with one to six halogens,
C₁₋₆ alkoxy, unsubstituted or substituted with one to six halogens,
C₃₋₆ cycloalkyl-C₀₋₆ alkylene, wherein cycloalkyl is unsubstituted or substituted with one to six substituents independently selected from halogen, cyano, C₁₋₆ alkyl and C₁₋₆ alkoxy, wherein alkyl and alkoxy are unsubstituted or substituted with one to six halogens, and
phenyl, wherein phenyl is unsubstituted or substituted with one to five substituents independently selected from halogen, cyano, C₁₋₆ alkyl and C₁₋₆alkoxy, wherein alkyl and alkoxy are unsubstituted or substituted with one to six halogens; and

### R⁵ is

—CH₂-phenyl, wherein phenyl is unsubstituted or substituted with one to five substituents independently selected from hydroxy, halogen, cyano, C₁₋₆ alkyl and C₁₋₆ alkoxy, wherein alkyl and alkoxy are unsubstituted or substituted with one to six halogens.

In a third embodiment of the compounds of the present invention, X═S as depicted in formula (If)

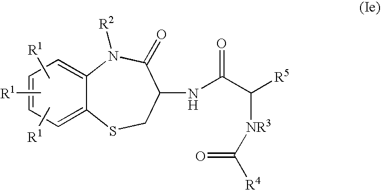

and pharmaceutically acceptable salts thereof, wherein R¹, R², R⁴ and R⁵ are as described above.

In a class of this embodiment of the compounds of the present invention, the carbon atom marked with an * has the stereochemical configuration as depicted in formula (Ig)

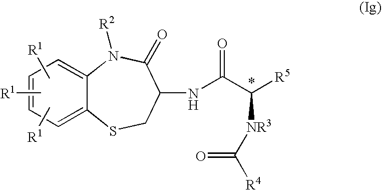

and pharmaceutically acceptable salts thereof, wherein R¹, R², R³, R⁴ and R⁵ are as defined herein.

Within this class, the invention encompasses compounds of formula (Ig) wherein:

R² is independently selected from the group consisting of
hydrogen,
C₁₋₆ alkyl, unsubstituted or substituted with one to six substituents selected from halogen and hydroxy,
C₁₋₆ alkenyl, and
C₁₋₆ alkoxy-C₁₋₆ alkylene, unsubstituted or substituted with one to six halogens;
   R³ is hydrogen;
   R⁴ is independently selected from the group consisting of
C₁₋₆ alkyl, unsubstituted or substituted with one to six halogens,
C₁₋₆ alkoxy, unsubstituted or substituted with one to six halogens,
C₃₋₆ cycloalkyl-C₀₋₆ alkylene, wherein cycloalkyl is unsubstituted or substituted with one to six substituents independently selected from halogen, cyano, C₁₋₆ alkyl and C₁₋₆ alkoxy, wherein alkyl and alkoxy are unsubstituted or substituted with one to six halogens, and
phenyl, wherein phenyl is unsubstituted or substituted with one to five substituents independently selected from halogen, cyano, C₁₋₆ alkyl and C₁₋₆ alkoxy, wherein alkyl and alkoxy are unsubstituted or substituted with one to six halogens; and

### R⁵ is

—CH₂-phenyl, wherein phenyl is unsubstituted or substituted with one to five substituents independently selected from hydroxy, halogen, cyano, C₁₋₆ alkyl and C₁₋₆ alkoxy, wherein alkyl and alkoxy are unsubstituted or substituted with one to six halogens.

Illustrative, but nonlimiting, examples of compounds of the present invention that are useful as sodium channel blockers are the following:

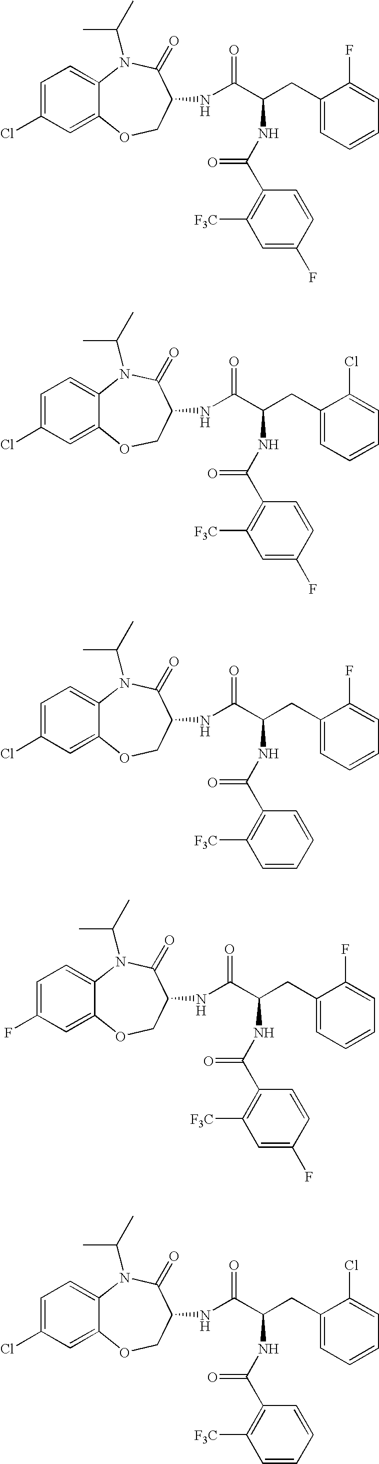

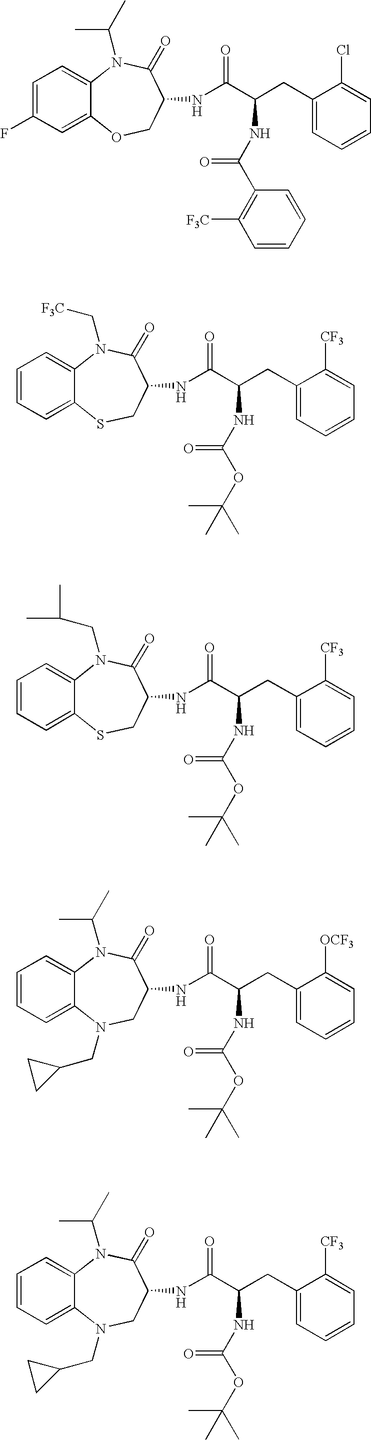

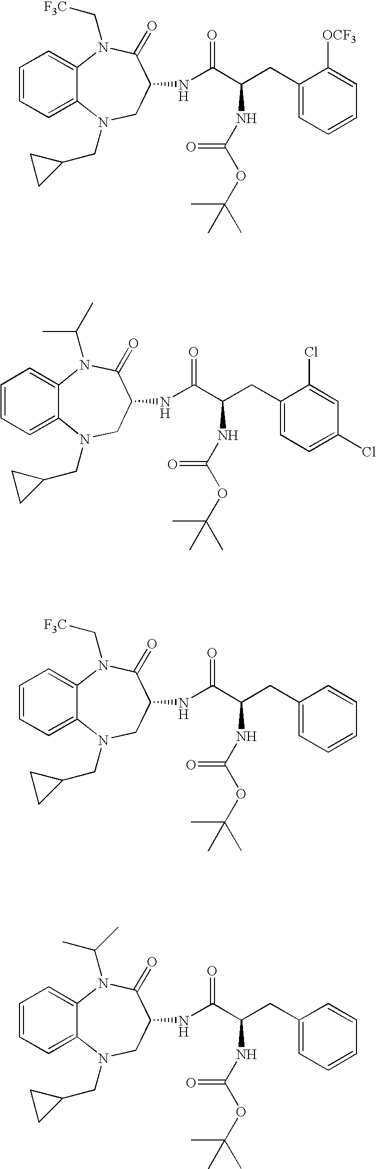

The invention also encompasses the examples described below.

The invention also encompasses a pharmaceutical composition comprising a therapeutically effective amount of a compound of formula (I), or a pharmaceutically acceptable salt thereof, and a pharmaceutically acceptable carrier.

The invention also encompasses a pharmaceutical composition comprising a therapeutically effective amount of a compound of formula (I), or a pharmaceutically acceptable salt thereof, and a pharmaceutically acceptable carrier and

further comprising a second therapeutic agent selected from the group consisting of: i) opiate agonists, ii) opiate antagonists, iii) calcium channel antagonists, iv) 5HT receptor agonists, v) 5HT receptor antagonists vi) sodium channel antagonists, vii) NMDA receptor agonists, viii) NMDA receptor antagonists, ix) COX-2 selective inhibitors, x) NK1 antagonists, xi) non-steroidal anti-inflammatory drugs, xii) selective serotonin reuptake inhibitors, xiii) selective serotonin and norepinephrine reuptake inhibitors, xiv) tricyclic antidepressant drugs, xv) norepinephrine modulators, xvi) lithium, xvii) valproate, xviii) neurontin, and xix) pregabalin.

The invention also encompasses a method of treatment or prevention of pain comprising the step of administering to a patient in need thereof a therapeutically effective amount, or a prophylactically effective amount, of a compound of formula (I), or a pharmaceutically acceptable salt thereof.

The invention also encompasses a method of treatment or prevention of one or more of the following condition in a patient in need thereof:

(1) chronic, visceral, inflammatory and/or neuropathic pain syndromes;

(2) pain resulting from, or associated with, traumatic nerve injury, nerve compression or entrapment, postherpetic neuralgia, trigeminal neuralgia, diabetic neuropathy, cancer and/or chemotherapy,

(3) chronic lower back pain;

(4) phantom limb pain; and

(5) HIV- and HIV treatment-induced neuropathy, chronic pelvic pain, neuroma pain, complex regional pain syndrome, chronic arthritic pain and related neuralgias; comprising the step of administering to a patient in need thereof a therapeutically effective amount, or a prophylactically effective amount, of a compound of formula (I), or a pharmaceutically acceptable salt thereof.

As used herein, “alkyl” as well as other groups having the prefix “alk” such as, for example, alkoxy, alkanoyl, alkenyl, and alkynyl means carbon chains which may be linear or branched or combinations thereof. Examples of alkyl groups include methyl, ethyl, propyl, isopropyl, butyl, sec- and tert-butyl, pentyl, hexyl, and heptyl. “Alkenyl,” “alkynyl” and other like terms include carbon chains containing at least one unsaturated C—C bond.

The term “cycloalkyl” refers to a saturated hydrocarbon containing one ring having a specified number of carbon atoms. Examples of cycloalkyl include cyclopropyl, cyclobutyl, cyclopentyl, and cyclohexyl. When “cycloalkyl” is substituted, it includes, for example, the following:

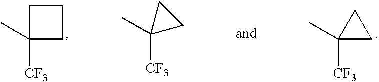

The term “C₀₋₄alkyl” includes alkyls containing 4, 3, 2, 1, or no carbon atoms. An alkyl with no carbon atoms is a hydrogen atom substituent when the alkyl is a terminal group and is a direct bond when the alkyl is a bridging group.

The term “alkoxy” refers to straight or branched chain alkoxides of the number of carbon atoms specified (e.g. C1-10 alkoxy) or any number within this range methoxy, ethoxy, isopropoxy, etc.).

“Aryl” means a mono or polycyclic aromatic ring system containing carbon ring atoms. The preferred aryls are mono or bicyclic 6-10 membered aromatic systems. Phenyl and naphthyl are preferred aryls. The most preferred is phenyl.

“Heteroaryl” means an aromatic or partially aromatic heterocycle that contains at least one ring heteroatom selected from O, S, and N. Heteroaryls also include heteroaryls fused to other kinds of rings such as aryls, cycloalkyls, and heterocycles that are not aromatic. Examples of heteroaryls include pyridinyl, quinolinyl, isoquinolinyl, pyridazinyl, pyrimidinyl, pyrazinyl, quinoxalinyl, furyl, benzofuryl, dibenzofuryl, thienyl, benzthienyl, pyrrolyl, indolyl, pyrazolyl, indazolyl, oxazolyl, benzoxazolyl, isoxazolyl, thiazolyl, benzothiazolyl, isothiazolyl, imidazolyl, benzimidazolyl, oxadiazolyl, thiadiazolyl, triazolyl, and tetrazolyl. Examples of heterocycloalkyls include azetidinyl, pyrrolidinyl, piperidinyl, piperazinyl, morpholinyl, tetrahydrofuranyl, imidazolinyl, pyrrolidin-2-one, piperidin-2-one, and thiomorpholinyl.

“Halogen” refers to fluorine, chlorine, bromine and iodine. Fluorine and Chlorine are generally preferred. Fluorine is most preferred when the halogens are substituted on an alkyl or alkoxy group ((e.g. CF₃O, CF₃CH₂O).

The term “mammal” “mammalian” or “mammals” includes humans, as well as animals, such as dogs, cats, horses, pigs and cattle.

Compounds described herein may contain one or more double bonds and may thus give rise to cis/trans isomers as well as other conformational isomers. The present invention includes all such possible isomers as well as mixtures of such isomers unless specifically stated otherwise.

The compounds of the present invention contain one or more asymmetric centers and may thus occur as racemates, racemic mixtures, single enantiomers, diastereomeric mixtures, and individual diastereomers. In particular the compounds of the present invention have an asymmetric center at the sterogenic carbon atoms marked with an * and ** in formulae Ib, Ie, and Ig. Additional asymmetric centers may be present depending upon the nature of the various substituents on the molecule. Each such asymmetric center will independently produce two optical isomers and it is intended that all of the possible optical isomers and diastereomers in mixtures and as pure or partially purified compounds are included within the ambit of this invention. The present invention is meant to comprehend all such isomeric forms of these compounds.

Formula I shows the structure of the class of compounds without preferred stereochemistry.

The independent syntheses of these diastereomers or their chromatographic separations may be achieved as known in the art by appropriate modification of the methodology disclosed herein. Their absolute stereochemistry may be determined by X-ray crystallography of crystalline products or crystalline intermediates which are derivatized, if necessary, with a reagent containing an asymmetric center of known absolute configuration.

If desired, racemic mixtures of the compounds may be separated so that the individual enantiomers are isolated. The separation can be carried out by methods well known in the art, such as by chromatographic methods utilizing chiral stationary phases.

Alternatively, any enantiomer of a compound may be obtained by stereoselective synthesis using optically pure starting materials or reagents of known configuration by methods well known in the art.

It will be understood that, as used herein, references to the compounds of structural formula I are meant to also include the pharmaceutically acceptable salts, and also salts that are not pharmaceutically acceptable when they are used as precursors to the free compounds or in other synthetic manipulations.

The compounds of the present invention may be administered in the form of a pharmaceutically acceptable salt. The term “pharmaceutically acceptable salts” refers to salts prepared from pharmaceutically acceptable non-toxic bases or acids. When the compound of the present invention is acidic, its corresponding salt can be conveniently prepared from pharmaceutically acceptable non-toxic bases, including inorganic bases and organic bases. Salts derived from such inorganic bases include aluminum, ammonium, calcium, copper (ic and ous), ferric, ferrous, lithium, magnesium, manganese (ic and ous), potassium, sodium, zinc and the like salts. Salts derived from pharmaceutically acceptable organic non-toxic bases include salts of primary, secondary, and tertiary amines, as well as cyclic amines and substituted amines such as naturally occurring and synthesized substituted amines. Other pharmaceutically acceptable organic non-toxic bases from which salts can be formed include ion exchange resins such as, for example, arginine, betaine, caffeine, choline, N,N′-dibenzylethylenediamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, and tromethamine.

When the compound of the present invention is basic, its corresponding salt can be conveniently prepared from pharmaceutically acceptable non-toxic acids, including inorganic and organic acids. Such acids include, for example, acetic, benzenesulfonic, benzoic, camphorsulfonic, citric, ethanesulfonic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric, p-toluenesulfonic acid and the like.

The pharmaceutical compositions of the present invention comprise compounds of the invention (or pharmaceutically acceptable salts thereof) as an active ingredient, a pharmaceutically acceptable carrier, and optionally one or more additional therapeutic agents or adjuvants. Such additional therapeutic agents can include, for example, i) opiate agonists or antagonists, ii) calcium channel antagonists, iii) 5HT receptor agonists or antagonists, iv) sodium channel antagonists, v) NMDA receptor agonists or antagonists, vi) COX-2 selective inhibitors, vii) NK1 antagonists, viii) non-steroidal anti-inflammatory drugs (“NSAID”), ix) selective serotonin reuptake inhibitors (“SSRI”) and/or selective serotonin and norepinephrine reuptake inhibitors (“SSNRI”), x) tricyclic antidepressant drugs, xi) norepinephrine modulators, xii) lithium, xiii) valproate, xiv) neurontin (gabapentin), and xv) pregabalin. The instant compositions include compositions suitable for oral, rectal, topical, and parenteral (including subcutaneous, intramuscular, and intravenous) administration, although the most suitable route in any given case will depend on the particular host, and nature and severity of the conditions for which the active ingredient is being administered. The pharmaceutical compositions may be conveniently presented in unit dosage form and prepared by any of the methods well known in the art of pharmacy.

The present compounds and compositions are useful for the treatment of chronic, visceral, inflammatory and neuropathic pain syndromes. They are useful for the treatment of pain resulting from traumatic nerve injury, nerve compression or entrapment, postherpetic neuralgia, trigeminal neuralgia, and diabetic neuropathy. The present compounds and compositions are also useful for the treatment of chronic lower back pain, phantom limb pain, chronic pelvic pain, neuroma pain, complex regional pain syndrome, chronic arthritic pain and related neuralgias, and pain associated with cancer, chemotherapy, HIV and HIV treatment-induced neuropathy. Compounds of this invention may also be utilized as local anesthetics. Compounds of this invention are useful for the treatment of irritable bowel syndrome and related disorders, as well as Crohn's disease.

The instant compounds have clinical uses for the treatment of epilepsy and partial and generalized tonic seizures. They are also useful for neuroprotection under ischemic conditions caused by stroke or neural trauma and for treating multiple sclerosis. The present compounds are useful for the treatment of tachy-arrhythmias. Additionally, the instant compounds are useful for the treatment of neuropsychiatric disorders, including mood disorders, such as depression or more particularly depressive disorders, for example, single episodic or recurrent major depressive disorders and dysthymic disorders, or bipolar disorders, for example, bipolar I disorder, bipolar II disorder and cyclothymic disorder; anxiety disorders, such as panic disorder with or without agoraphobia, agoraphobia without history of panic disorder, specific phobias, for example, specific animal phobias, social phobias, obsessive-compulsive disorder, stress disorders including post-traumatic stress disorder and acute stress disorder, and generalized anxiety disorders.

In addition to primates, such as humans, a variety of other mammals can be treated according to the method of the present invention. For instance, mammals including, but not limited to, cows, sheep, goats, horses, dogs, cats guinea pigs, or other bovine, ovine, equine, canine, feline, rodent such as mouse, species can be treated. However, the method can also be practiced in other species, such as avian species (e.g., chickens).

It will be appreciated that for the treatment of depression or anxiety, a compound of the present invention may be used in conjunction with other anti-depressant or anti-anxiety agents, such as norepinephrine reuptake inhibitors, selective serotonin reuptake inhibitors (SSRIs), monoamine oxidase inhibitors (MAOIs), reversible inhibitors of monoamine oxidase (RIMAs), serotonin and noradrenaline reuptake inhibitors (SNRIs), α-adrenoreceptor antagonists, atypical anti-depressants, benzodiazepines, 5-HT_{1A} agonists or antagonists, especially 5-HT_{1A} partial agonists, neurokinin-1 receptor antagonists, corticotropin releasing factor (CRF) antagonists, and pharmaceutically acceptable salts thereof.

Further, it is understood that compounds of this invention can be administered at prophylactically effective dosage levels to prevent the above-recited conditions and disorders, as well as to prevent other conditions and disorders associated with sodium channel activity.

Creams, ointments, jellies, solutions, or suspensions containing the instant compounds can be employed for topical use. Mouth washes and gargles are included within the scope of topical use for the purposes of this invention.

Dosage levels from about 0.01 mg/kg to about 140 mg/kg of body weight per day are useful in the treatment of inflammatory and neuropathic pain, or alternatively about 0.5 mg to about 7 g per patient per day. For example, inflammatory pain may be effectively treated by the administration of from about 0.01 mg to about 75 mg of the compound per kilogram of body weight per day, or alternatively about 0.5 mg to about 3.5 g per patient per day. Neuropathic pain may be effectively treated by the administration of from about 0.01 mg to about 125 mg of the compound per kilogram of body weight per day, or alternatively about 0.5 mg to about 5.5 g per patient per day.

The amount of active ingredient that may be combined with the carrier materials to produce a single dosage form will vary depending upon the host treated and the particular mode of administration. For example, a formulation intended for the oral administration to humans may conveniently contain from about 0.5 mg to about 5 g of active agent, compounded with an appropriate and convenient amount of carrier material which may ary from about 5 to about 95 percent of the total composition. Unit dosage forms will generally contain between from about 1 mg to about 1000 mg of the active ingredient, typically 25 mg, 50 mg, 100 mg, 200 mg, 300 mg, 400 mg, 500 mg, 600 mg, 800 mg or 1000 mg.

It is understood, however, that the specific dose level for any particular patient will depend upon a variety of factors. Such patient-related factors include the age, body weight, general health, sex, and diet of the patient. Other factors include the time and route of administration, rate of excretion, drug combination, and the severity of the particular disease undergoing therapy.

In practice, the compounds of the invention, or pharmaceutically acceptable salts thereof, can be combined as the active ingredient in intimate admixture with a pharmaceutical carrier according to conventional pharmaceutical compounding techniques. The carrier may take a wide variety of forms depending on the form of preparation desired for administration, e.g., oral or parenteral (including intravenous). Thus, the pharmaceutical compositions of the present invention can be presented as discrete units suitable for oral administration such as capsules, cachets or tablets each containing a predetermined amount of the active ingredient. Further, the compositions can be presented as a powder, as granules, as a solution, as a suspension in an aqueous liquid, as a non-aqueous liquid, as an oil-in-water emulsion or as a water-in-oil liquid emulsion. In addition to the common dosage forms set out above, the compounds of the invention, or pharmaceutically acceptable salts thereof, may also be administered by controlled release means and/or delivery devices. The compositions may be prepared by any of the methods of pharmacy. In general, such methods include a step of bringing into association the active ingredient with the carrier that constitutes one or more necessary ingredients. In general, the compositions are prepared by uniformly and intimately admixing the active ingredient with liquid carriers or finely divided solid carriers or both. The product can then be conveniently shaped into the desired presentation.

Thus, the pharmaceutical compositions of this invention may include a pharmaceutically acceptable carrier and a compound or a pharmaceutically acceptable salt of Formula I, Ia, Ib, Ic, Id, Ie, If or Ig. The compounds of the invention, or pharmaceutically acceptable salts thereof, can also be included in pharmaceutical compositions in combination with one or more therapeutically active compounds.

The pharmaceutical carrier employed can be, for example, a solid, liquid, or gas. Examples of solid carriers include lactose, terra alba, sucrose, talc, gelatin, agar, pectin, acacia, magnesium stearate, and stearic acid. Examples of liquid carriers are sugar syrup, peanut oil, olive oil, and water. Examples of gaseous carriers include carbon dioxide and nitrogen.

In preparing the compositions for oral dosage form, any convenient pharmaceutical media may be employed. For example, water, glycols, oils, alcohols, flavoring agents, preservatives, coloring agents and the like may be used to form oral liquid preparations such as suspensions, elixirs and solutions; while carriers such as starches, sugars, microcrystalline cellulose, diluents, granulating agents, lubricants, binders, and disintegrating agents can be used to form oral solid preparations such as powders, capsules and tablets. Because of their ease of administration, tablets and capsules are the preferred oral dosage units whereby solid pharmaceutical carriers are employed. Optionally, tablets may be coated by standard aqueous or nonaqueous techniques

A tablet containing the composition of this invention may be prepared by compression or molding, optionally with one or more accessory ingredients or adjuvants. Compressed tablets may be prepared by compressing, in a suitable machine, the active ingredient in a free-flowing form such as powder or granules, optionally mixed with a binder, lubricant, inert diluent, surface active or dispersing agent. Molded tablets may be made by molding in a suitable machine, a mixture of the powdered compound moistened with an inert liquid diluent. Each tablet preferably contains from about 0.1 mg to about 500 mg of the active ingredient and each cachet or capsule preferably containing from about 0.1 mg to about 500 mg of the active ingredient. Thus, a tablet, cachet, or capsule conveniently contains 0.1 mg, 1 mg, 5 mg, 25 mg, 50 mg, 100 mg, 200 mg, 300 mg, 400 mg, or 500 mg of the active ingredient taken one or two tablets, cachets, or capsules, once, twice, or three times daily.

Pharmaceutical compositions of the present invention suitable for parenteral administration may be prepared as solutions or suspensions of the active compounds in water. A suitable surfactant can be included such as, for example, hydroxypropylcellulose. Dispersions can also be prepared in glycerol, liquid polyethylene glycols, and mixtures thereof in oils. Further, a preservative can be included to prevent the detrimental growth of microorganisms.

Pharmaceutical compositions of the present invention suitable for injectable use include sterile aqueous solutions or dispersions. Furthermore, the compositions can be in the form of sterile powders for the extemporaneous preparation of such sterile injectable solutions or dispersions. In all cases, the final injectable form must be sterile and must be effectively fluid for easy syringability. The pharmaceutical compositions must be stable under the conditions of manufacture and storage, and thus should be preserved against the contaminating action of microorganisms such as bacteria and fungi. The carrier can be a solvent or dispersion medium containing, for example, water, ethanol, polyol (e.g. glycerol, propylene glycol and liquid polyethylene glycol), vegetable oils, and suitable mixtures thereof.

Pharmaceutical compositions of the present invention can be in a form suitable for topical use such as, for example, an aerosol, cream, ointment, lotion, and dusting powder. Further, the compositions can be in a form suitable for use in transdermal devices. These formulations may be prepared, utilizing a compound represented of the invention, or pharmaceutically acceptable salts thereof, via conventional processing methods. As an example, a cream or ointment is prepared by mixing hydrophilic material and water, together with about 5 wt % to about 10 wt % of the compound, to produce a cream or ointment having a desired consistency.

Pharmaceutical compositions of this invention can be in a form suitable for rectal administration wherein the carrier is a solid, such as, for example, where the mixture forms unit dose suppositories. Suitable carriers include cocoa butter and other materials commonly used in the art. The suppositories may be conveniently formed by first admixing the composition with the softened or melted carrier(s) followed by chilling and shaping in moulds.

In addition to the aforementioned carrier ingredients, the pharmaceutical formulations described above may include, as appropriate, one or more additional carrier ingredients such as diluents, buffers, flavoring agents, binders, surface-active agents, thickeners, lubricants, and preservatives (including anti-oxidants). Furthermore, other adjuvants can be included to render the formulation isotonic with the blood of the intended recipient. Compositions containing a compound of the invention, or pharmaceutically acceptable salts thereof, can also be prepared in powder or liquid concentrate form.

The compounds and pharmaceutical compositions of this invention have been found to block sodium channels. Accordingly, an aspect of the invention is the treatment and prevention in mammals of conditions that are amenable to amelioration through blockage of neuronal sodium channels by administering an effective amount of a compound of this invention. Such conditions include, for example, acute pain, chronic pain, visceral pain, inflammatory pain and neuropathic pain. The instant compounds and compositions are useful for treating and preventing the above-recited conditions, including acute pain, chronic pain, visceral pain, inflammatory pain and neuropathic pain, in humans and non-human mammals such as dogs and cats. It is understood that the treatment of mammals other than humans refers to the treatment of clinical conditions in non-human mammals that correlate to the above-recited conditions.

Further, as described above, the instant compounds can be utilized in combination with one or more therapeutically active compounds. In particular, the inventive compounds can be advantageously used in combination with i) opiate agonists or antagonists, ii) calcium channel antagonists, iii) 5HT receptor agonists or antagonists, including 5-HT_{1A} agonists or antagonists, and 5-HT_{1A} partial agonists, iv) sodium channel antagonists, v) N-methyl-D-aspartate (NMDA) receptor agonists or antagonists, vi) COX-2 selective inhibitors, vii) neurokinin receptor 1 (NK1) antagonists, viii) non-steroidal anti-inflammatory drugs (NSAID), ix) selective serotonin reuptake inhibitors (SSRI) and/or selective serotonin and norepinephrine reuptake inhibitors (SSNRI), x) tricyclic antidepressant drugs, xi) norepinephrine modulators, xii) lithium, xiii) valproate, xiv) norepinephrine reuptake inhibitors, xv) monoamine oxidase inhibitors (MAOIs), xvi) reversible inhibitors of monoamine oxidase (RIMAs), xvi) α-adrenoreceptor antagonists, xviii) atypical anti-depressants, xix) benzodiazepines, xx) corticotropin releasing factor (CRF) antagonists, xxi) neurontin (gabapentin), and xxii) pregabalin.

The abbreviations used herein have the following meanings (abbreviations not shown here have their meanings as commonly used unless specifically stated otherwise): Ac (acetyl), Bn (benzyl), Boc (tertiary-butoxy carbonyl), CAMP (cyclic adenosine-3′,5′-monophosphate), DAST ((diethylamino)sulfur trifluoride), DBU (1,8-diazabicyclo[5.4.0]undec-7-ene), DIBAL (diisobutylaluminum hydride), DMAP (4-(dimethylamino)pyridine), DMF (N,N-dimethylformamide), EDC (1-(3-dimethylaminopropyl)-3-ethylcarbodiimide hydrochloride), Et₃N (triethylamine), GST (glutathione transferase), HOBt (1-hydroxybenzotriazole), LAH (lithium aluminum hydride), Ms (methanesulfonyl; mesyl; or SO₂Me), MsO (methanesulfonate or mesylate), NBS (N-bromosuccinimide), NCS(N-chlorosuccinimide), NSAID (non-steroidal anti-inflammatory drug), PDE (Phosphodiesterase), Ph (Phenyl), r.t. or RT (room temperature), Rac (Racemic), SAM (aminosulfonyl; sulfonamide or SO₂NH₂), SPA (scintillation proximity assay), Th (2- or 3-thienyl), TFA (trifluoroacetic acid), THF (Tetrahydrofuran), Thi (Thiophenediyl), TLC (thin layer chromatography), TMEDA (N,N,N′,N′-tetramethylethylenediamine), TMSI (trimethylsilyl iodide), Tr or trityl (N-triphenylmethyl), C₃H₅ (Allyl), Me (methyl), Et (ethyl), n-Pr (normal propyl), i-Pr (isopropyl), n-Bu (normal butyl), i-Butyl (isobutyl), s-Bu (secondary butyl), t-Bu (tertiary butyl), c-Pr (cyclopropyl), c-Bu (cyclobutyl), c-Pen (cyclopentyl), c-Hex (cyclohexyl).

The present compounds can be prepared according to the general Schemes provided below as well as the procedures provided in the Examples. The following Schemes and Examples further describe, but do not limit, the scope of the invention.

Unless specifically stated otherwise, the experimental procedures were performed under the following conditions: All operations were carried out at room or ambient temperature; that is, at a temperature in the range of 18-25° C. Evaporation of solvent was carried out using a rotary evaporator under reduced pressure (600-4000 pascals: 4.5-30 mm Hg) with a bath temperature of up to 60° C. The course of reactions was followed by thin layer chromatography (TLC) or by high-pressure liquid chromatography-mass spectrometry (HPLC-MS), and reaction times are given for illustration only. The structure and purity of all final products were assured by at least one of the following techniques: TLC, mass spectrometry, nuclear magnetic resonance (NMR) spectrometry or microanalytical data. When given, yields are for illustration only. When given, NMR data is in the form of delta (δ) values for major diagnostic protons, given in parts per million (ppm) relative to tetramethylsilane (TMS) as internal standard, determined at 300 MHz, 400 MHz or 500 MHz using the indicated solvent. Conventional abbreviations used for signal shape are: s. singlet; d. doublet; t. triplet; m. multiplet; br. Broad; etc. In addition, “Ar” signifies an aromatic signal. Chemical symbols have their usual meanings; the following abbreviations are used: v (volume), w (weight), b.p. (boiling point), m.p. (melting point), L (liter(s)), mL (milliliters), g (gram(s)), mg (milligrams(s)), mol (moles), mmol (millimoles), eq (equivalent(s)).

### Methods of Synthesis

Compounds of the present invention can be prepared according to the Schemes provided below as well as the procedures provided in the Examples. The substituents are the same as in the above Formulas except where defined otherwise or otherwise apparent to the ordinary skilled artisan.

The novel compounds of the present invention can be readily synthesized using techniques known to those skilled in the art, such as those described, for example, in Advanced Organic Chemistry, March, 5th Ed., John Wiley and Sons, New York, N.Y., 2001; Advanced Organic Chemistry, Carey and Sundberg, Vol. A and B, 3^{rd} Ed., Plenum Press, Inc., New York, N.Y., 1990; Protective groups in Organic Synthesis, Green and Wuts, 2^{nd} Ed., John Wiley and Sons, New York, N.Y., 1991; Comprehensive Organic Transformations, Larock, VCH Publishers, Inc., New York, N.Y., 1988; Handbook of Heterocyclic Chemistry, Katritzky and Pozharskii, 2^{nd} Ed., Pergamon, New York, N.Y., 2000 and references cited therein. The starting materials for the present compounds may be prepared using standard synthetic transformations of chemical precursors that are readily available from commercial sources, including Aldrich Chemical Co. (Milwaukee, Wis.); Sigma Chemical Co. (St. Louis, Mo.); Lancaster Synthesis (Windham, N.H.); Ryan Scientific (Columbia, S.C.); Maybridge (Cornwall, UK); Matrix Scientific (Columbia, S.C.); Arcos, (Pittsburgh, Pa.) and Trans World Chemicals (Rockville, Md.).

The procedures described herein for synthesizing the compounds may include one or more steps of protecting group manipulations and of purification, such as, recrystallization, distillation, column chromatography, flash chromatography, thin-layer chromatography (TLC), radial chromatography and high-pressure liquid chromatography (HPLC). The products can be characterized using various techniques well known in the chemical arts, including proton and carbon-13 nuclear magnetic resonance (¹H and ¹³C NMR), infrared and ultraviolet spectroscopy (IR and UV), X-ray crystallography, elemental analysis and HPLC and mass spectrometry (HPLC-MS). Methods of protecting group manipulation, purification, structure identification and quantification are well known to one skilled in the art of chemical synthesis.

Appropriate solvents are those which will at least partially dissolve one or all of the reactants and will not adversely interact with either the reactants or the product. Suitable solvents are aromatic hydrocarbons (e.g, toluene, xylenes), halogenated solvents (e.g, methylene chloride, chloroform, carbontetrachloride, chlorobenzenes), ethers (e.g, diethyl ether, diisopropylether, tert-butyl methyl ether, diglyme, tetrahydrofuran, dioxane, anisole), nitriles (e.g, acetonitrile, propionitrile), ketones (e.g, 2-butanone, dithyl ketone, tert-butyl methyl ketone), alcohols (e.g, methanol, ethanol, n-propanol, iso-propanol, n-butanol, t-butanol), N,N-dimethyl formamide (DMF), dimethylsulfoxide (DMSO) and water. Mixtures of two or more solvents can also be used. Suitable bases are, generally, alkali metal hydroxides, alkaline earth metal hydroxides such as lithium hydroxide, sodium hydroxide, potassium hydroxide, barium hydroxide, and calcium hydroxide; alkali metal hydrides and alkaline earth metal hydrides such as lithium hydride, sodium hydride, potassium hydride and calcium hydride; alkali metal amides such as lithium amide, sodium amide and potassium amide; alkali metal carbonates and alkaline earth metal carbonates such as lithium carbonate, sodium carbonate, cesium carbonate, sodium hydrogen carbonate, and cesium hydrogen carbonate; alkali metal alkoxides and alkaline earth metal alkoxides such as sodium methoxide, sodium ethoxide, potassium tert-butoxide and magnesium ethoxide; alkali metal alkyls such as methyllithium, n-butyllithium, sec-butyllithium, t-bultyllithium, phenyllithium, alkyl magnaesium halides, organic bases such as trimethylamine, triethylamine, triisopropylamine, N,N-diisopropylethylamine, piperidine, N-methyl piperidine, morpholine, N-methyl morpholine, pyridine, collidines, lutidines, and 4-dimethylaminopyridine; and bicyclic amines such as DBU and DABCO.

As described previously, in preparing the compositions for oral dosage form, any of the usual pharmaceutical media can be employed. For example, in the case of oral liquid preparations such as suspensions, elixirs and solutions, water, glycols, oils, alcohols, flavoring agents, preservatives, coloring agents and the like may be used; or in the case of oral solid preparations such as powders, capsules and tablets, carriers such as starches, sugars, microcrystalline cellulose, diluents, granulating agents, lubricants, binders, disintegrating agents, and the like may be included. Because of their ease of administration, tablets and capsules represent the most advantageous oral dosage unit form in which solid pharmaceutical carriers are employed. If desired, tablets may be coated by standard aqueous or nonaqueous techniques. In addition to the common dosage forms set out above, controlled release means and/or delivery devices may also be used in administering the instant compounds and compositions.

It is understood that the functional groups present in compounds described in the Schemes below can be further manipulated, when appropriate, using the standard functional group transformation techniques available to those skilled in the art, to provide desired compounds described in this invention.

It is also understood that compounds listed in the Schemes and Tables below that contain one or more stereocenters may be prepared as single enantiomers or diastereomers, or as mixtures containing two or more enantiomers or diastereomers in any proportion.

Other variations or modifications, which will be obvious to those skilled in the art, are within the scope and teachings of this invention. This invention is not to be limited except as set forth in the following claims.

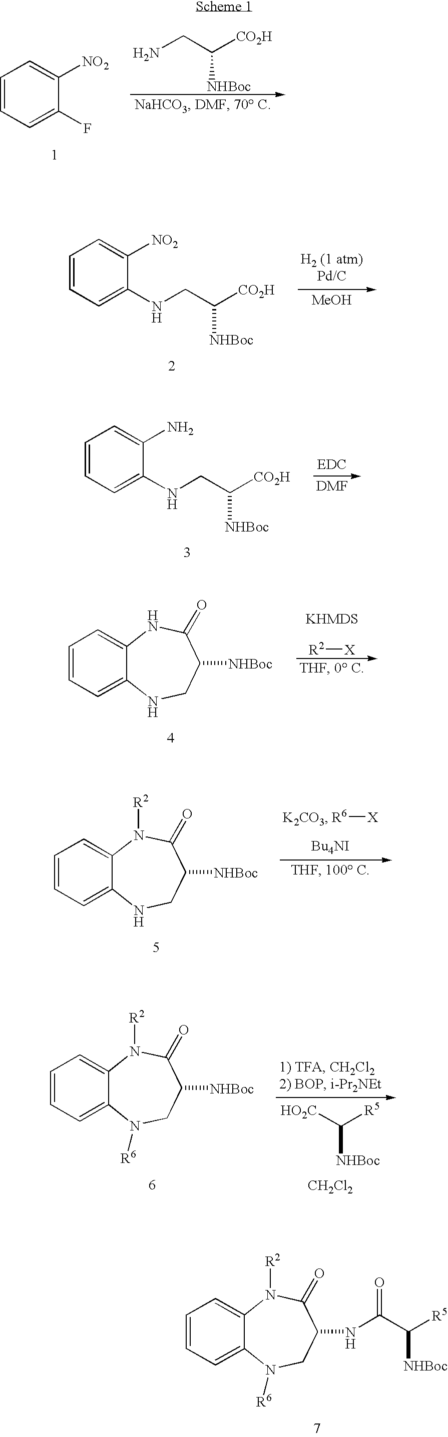

Scheme 1 summarizes one protocol for the preparation of compounds of formula Ia. The initial starting material, 1-fluoro-2-nitrobenzene 1, could be converted to intermediate 4 via established procedures [Lauffer, D. J., Mullican, M. D. A Practical Synthesis of (S)-3-tert-Butoxycarbonylamino-2-oxo-2,3,4,5-tetrahydro-1,5-benzodiazepine-1-acetic Acid Methyl Ester as a Conformationally Restricted Dipeptido-Mimetic for Caspase-1 (ICE) Inhibitors. Bioorganic & Medicinal Chemistry Letters 12, 1225-1227 (2002)]. Thus, a mixture of 1-fluoro-2-nitrobenzene 1, (R)-3-amino-2-tert-butoxycarbonylamino-propionic acid and a base such as sodium bicarbonate (NaHCO₃) could be heated in a solvent such as N,N-dimethylformamide (DMF) to provide aromatic substitution product 2. A solution of 2 in a solvent such as methanol (MeOH) could then be stirred under an atmosphere of hydrogen in the presence of a catalyst such as Pd/C to give aniline 3. Upon exposure to an activating agent such as 143-(dimethylamino)propyl]-3-ethylcarbodiimide hydrochloride (EDC) in a solvent such as N,N-dimethylformamide (DMF), compound 3 can undergo intramolecular cyclization to give benzodiazepinone 4. A solution of 4 in a solvent such as tetrahydrofuran (THF) could be cooled to 0° C. and treated first with a base such as potassium bis(trimethylsilyl)amide (KHMDS) and then with an electrophile R²—X wherein X is a halide or triflate to give alkylated product 5. A mixture of 5, a base such as potassium carbonate (K₂CO₃), an electrophile R⁶—X wherein X is a halide or triflate, and a catalyst such as tetrabutylammonium iodide (Bu₄NI) could be heated in a solvent such as tetrahydrofuran (THF) at temperatures ranging from 60° C. to 100° C. to provide compound 6. Finally, the N-Boc protecting group of 6 could be removed by reaction with an acid such as trifluoroacetic acid (TFA) in a solvent such as dichloromethane (CH₂Cl₂) to give the corresponding amine. This amine could then be coupled with an N-Boc protected D-amino acid in the presence of an activating agent such as benzotriazol-1-yloxytris(dimethylamino)phosphonium hexafluorophosphate (BOP) and a base such as diisopropylethylamine to give coupled product 7. The amino acids used in this coupling reaction could either be obtained from commercial sources or synthesized via the method of Williams and coworkers [Williams, R. M., Myeong-Nyeo, I. Asymmetric synthesis of monosubstituted and alpha, alpha-disubstituted alpha-amino acids via diastereoselective glycine enolate alkylations. Journal of the American Chemical Society 113, 9276-9286 (1991)] or the method of Schollkopf [Schollkopf, U. Enantioselective synthesis of non-proteinogenic amino acids via metallated bis-lactim ethers of 2,5-diketopiperazines. Tetrahedron 39, 2085-2091 (1983), and references contained therein].

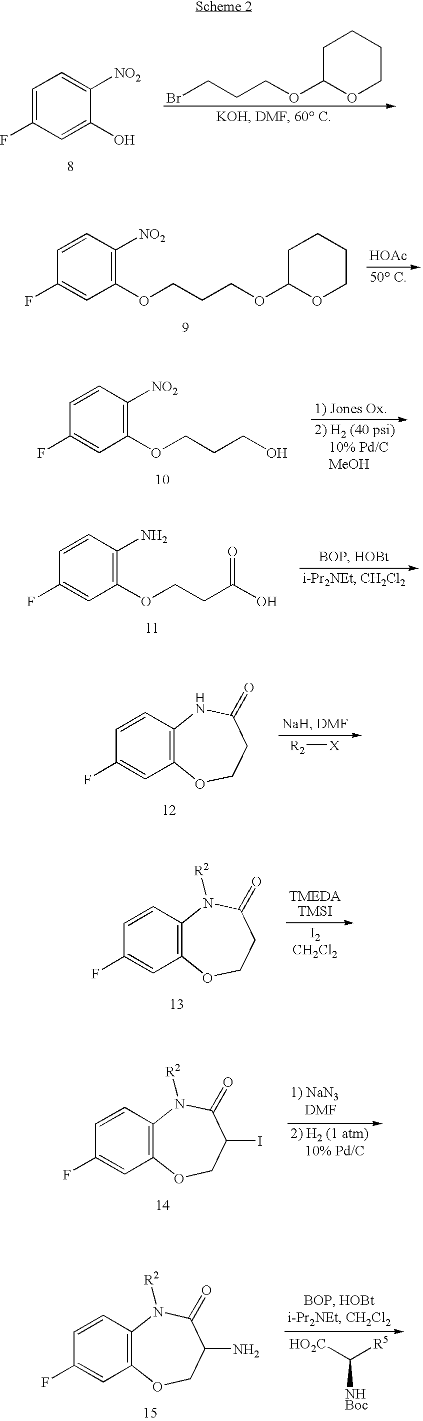

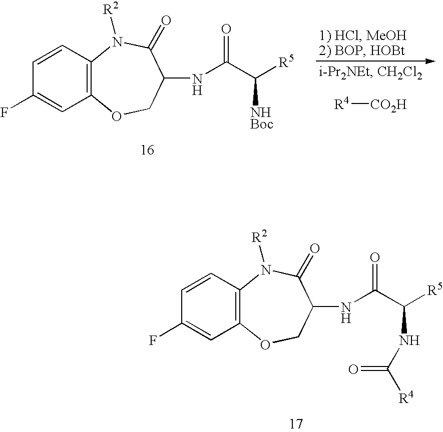

One method for the synthesis of compounds of formula Id or Ie is summarized in Scheme 2. The initial starting material, commercially available 5-fluoro-2-nitrophenol 8, could be heated in the presence of 2-(3-bromopropoxy)tetrahydro-2H-pyran and a base such as potassium hydroxide (KOH) to yield adduct 9. Warming of a solution of 9 in an acid such as acetic acid (HOAc) then induces hydroxyl deprotection, thereby providing 10. Exposure of 10 to an oxidizing reagent such as the Jones reagent effects conversion to the corresponding carboxylic acid derivative; subsequent hydrogenation of that species at 40 psi using a catalyst such as 10% Pd/C then furnishes aniline 11. Upon treatment with an activating agent such as benzotriazol-1-yloxytris(dimethylamino)phosphonium hexafluorophosphate (BOP), 1-hydroxybenzotriazole (HOBt), and a base such as diisopropylethylamine, 11 can undergo intramolecular cyclization to yield benzoxazepinone 12.

Once the benzoxazepinone ring system is constructed, it can be functionalized in a variety of ways. For instance, a solution of 12 in a solvent such as N,N-dimethylformamide (DMF) could be treated first with a base such as sodium hydride (NaH) and then with an electrophile R²—X wherein X is halide or triflate to yield alkylated product 13. Compound 13 could then be elaborated using a modified version of the process developed by Armstrong and coworkers [Armstrong, J. D., Eng, K. K., Keller, J. L., Purick, R. M., Hartner, F. W., Choi, W-B., Askin, D., Volante, R. P. An efficient asymmetric synthesis of (R)-3-amino-2,3,4,5-tetrahydro-1H-[1]-benzazepin-2-one. Tetrahedron Letters 35, 3239-3242 (1994)]. Thus, a cooled solution of 13 in a solvent such as dichloromethane could be treated sequentially with N,N,N′,N′-tetramethylethylenediamine (TMEDA), iodotrimethylsilane (TMSI) and iodine to yield alpha-iodinated product 14. Mild heating of 14 in a solvent such as N,N-dimethylformamide (DMF) in the presence of sodium azide could then result in displacement of the iodide to give the corresponding alpha-azido derivative. Reductive hydrogenation of the alpha-azide in the presence of a catalyst such as Pd/C could then provide amine 15. This amine could subsequently be coupled with an N-Boc protected D-amino acid in the presence of an activating agent such as benzotriazol-1-yloxytris(dimethylamino)phosphonium hexafluorophosphate (BOP), 1-hydroxybenzotriazole (HOBt), and a base such as diisopropylethylamine, thereby furnishing coupled product 16 as a mixture of diastereomers. If desired, the diastereomeric mixture could be purified via high-pressure liquid chromatography (HPLC) using a chiral column to give enantioenriched material. Note that the amino acids used in this coupling reaction could either be obtained from commercial sources or synthesized via the methods of Williams or Schollkopf noted above. Finally, exposure of 16 to an acid such as HCl in a solvent such as methanol (MeOH) could result in N-Boc deprotection to afford the corresponding amine. That amine could then be coupled with a commercially available carboxylic acid R⁴—CO₂H using conditions described above to give coupled product 17.

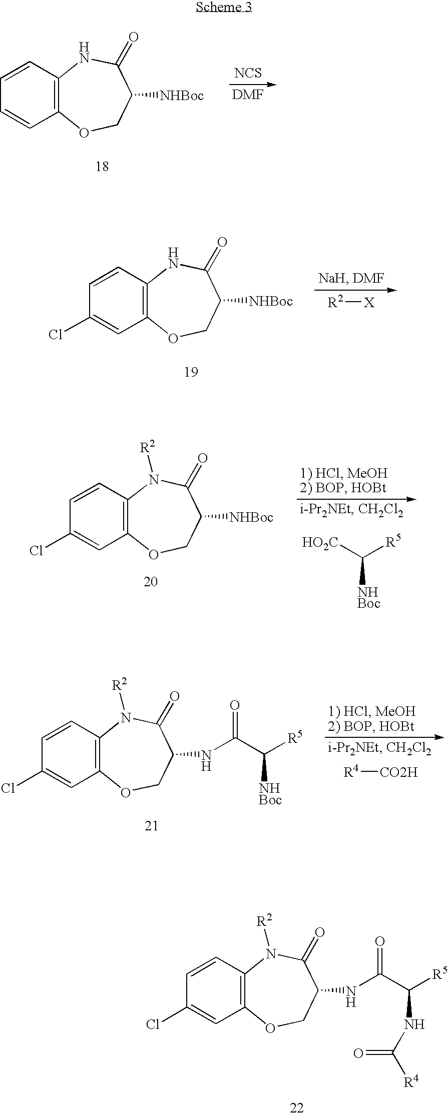

An additional method for the preparation of compounds of formula Id or Ie is outlined in Scheme 3. Starting material 18 can be prepared via a known procedure [DeVita, R. J., Schoen, W. R., Doldouras, G. A., Fisher, M. H., Wyvratt, M. J., Cheng, K., Chan, W. W.-S., Butler, B. S., Smith, R. G. Heterocyclic Analogs of the Benzolactam Nucleus of the Non-Peptidic Growth Hormone Secretagogue L-692,429. Bioorganic & Medicinal Chemistry Letters 5, 1281-1286 (1995)]. Exposure of 18 to a chlorinating reagent such as N-chlorosuccinimide (NCS) in a solvent such as N,N-dimethylformamide (DMF) can result in regioselective chlorination to give 19. A solution of 19 in a solvent such as N,N-dimethylformamide (DMF) could be treated first with a base such as sodium hydride (NaH) and then with an electrophile R²—X wherein X is halide or triflate to yield alkylated product 20. The N-Boc amine protecting group present in 20 could then be removed using acidic conditions. Thus, exposure of 20 to a solution of an acid such as HCl in a solvent such as methanol (MeOH) resulted in deprotection to give the corresponding amine. This amine could subsequently be coupled with an N-Boc protected D-amino acid in the presence of an activating agent such as benzotriazol-1-yloxytris(dimethylamino)phosphonium hexafluorophosphate (BOP), 1-hydroxybenzotriazole (HOBt), and a base such as diisopropylethylamine in a solvent such as dichloromethane, thereby providing coupled product 21. The amino acids used in this coupling reaction could either be obtained from commercial sources or synthesized via the methods of Williams or Schollkopf noted above. Exposure of 21 to an acid such as HCl in a solvent such as methanol (MeOH) resulted in N-Boc deprotection to afford the corresponding amine. This amine could then be coupled with a commercially available carboxylic acid R⁴—CO₂H using conditions described above to give coupled product 22.

Note that ((S)-8-oxo-6,7,8,9-tetrahydro-5-oxa-9-aza-benzocyclohepten-7-yl)-carbamic acid tert-butyl ester, the enantiomer of starting material 18, could be prepared via the method of Itoh and coworkers[Itoh, K., Kori, M., Inada, Y., Nishikawa, K., Kawamatsu, Y., Sugihara, H. Chemical & Pharmaceutical Bulletin, 34, 1128 (1986)]. Once synthesized, it could then be processed as described above to yield analogs of 22 with S stereochemistry at the 7-position.

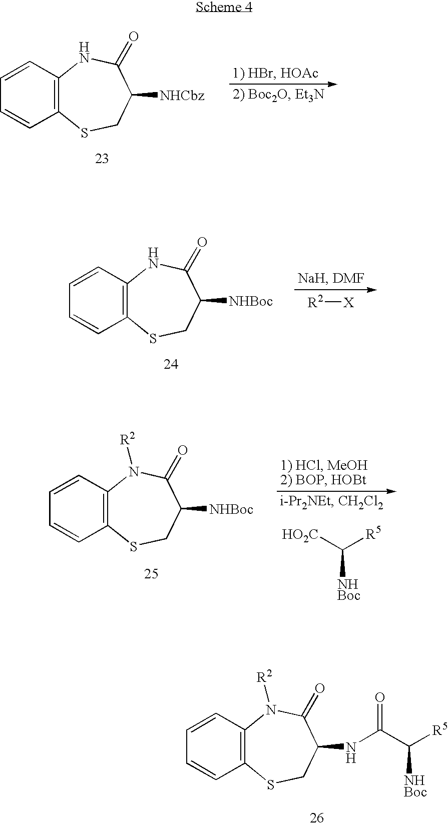

A method for the synthesis of compounds of formula If or Ig is outlined in Scheme 4. The initial starting material, ((R)-4-oxo-2,3,4,5-tetrahydro-benzo[b][1,4]thiazepin-3-yl)-carbamic acid benzyl ester 23, could be prepared via known procedures [Slade, J., Stanton, J. L., Ben-David, D., Mazzenga, G. C. Angiotensin converting enzyme inhibitors: 1,5-benzothiazepine derivatives. Journal of Medicinal Chemistry 28, 1517-1521 (1985)]. Exposure of 23 to an acid such as hydrogen bromide (HBr) in a solvent such as acetic acid (HOAc) could effect removal of the benzyloxycarbonyl (CBz) protecting group, thereby providing the corresponding amine. Reaction of that amine with di-tert-butylcarbonate (Boc₂O) in the presence of a base such as triethylamine could then yield the Boc-protected species 24. A solution of 24 in a solvent such as N,N-dimethylformamide (DMF) could be treated first with a base such as sodium hydride (NaH) and subsequently with an electrophile R²—X wherein X is halide or triflate to yield alkylated product 25. The N-Boc amine protecting group present in 25 could then be removed using acidic conditions. Thus, exposure of 25 to a solution of an acid such as HCl in a solvent such as methanol (MeOH) resulted in deprotection to give the corresponding amine. That amine could subsequently be coupled with an N-Boc protected D-amino acid in the presence of an activating agent such as 1-[3-(dimethylamino)propyl]-3-ethylcarbodiimide hydrochloride (EDC), 1-hydroxybenzotriazole (HOBt), and a base such as diisopropylethylamine, thereby providing coupled product 26. The amino acids used in this coupling reaction could either be obtained from commercial sources or synthesized via the methods of Williams or Schollkopf noted above.

Note that ((S)-4-oxo-2,3,4,5-tetrahydro-benzo[b][1,4]thiazepin-3-yl)-carbamic acid benzyl ester, the enantiomer of starting material 23, could be prepared via the method of DeVita and coworkers [DeVita, R. J., Schoen, W. R., Doldouras, G. A., Fisher, M. H., Wyvratt, M. J., Cheng, K., Chan, W. W.-S., Butler, B. S., Smith, R. G. Heterocyclic Analogs of the Benzolactam Nucleus of the Non-Peptidic Growth Hormone Secretagogue L-692,429. Bioorganic & Medicinal Chemistry Letters 5, 1281-1286 (1995)]. Once synthesized, it could then be processed as described above to yield analogs of 26 with S stereochemistry at the 3-position.

### Example 1

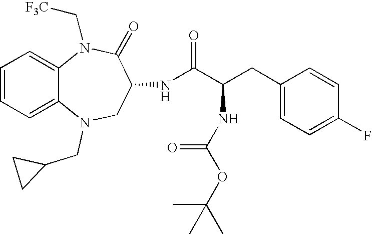

### [(R)-1-[(R)-5-Cyclopropylmethyl-2-oxo-1-(2,2,2-trifluoro-ethyl)-2,3,4,5-tetrahydro-1H-benzo[b][1,4]diazepin-3-ylcarbamoyl]-2-(4-fluoro-phenyl)-ethyl]-carbamic acid tert-butyl ester

### Step 1: Preparation of ((R)-2-oxo-2,3,4,5-tetrahydro-1H-benzo[b][1,4]diazepin-3-yl)-carbamic acid tert-butyl ester

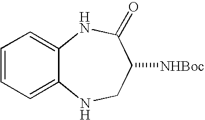

A mixture of 1-fluoro-2-nitrobenzene (7.77 g, 55.1 mmol), (R)-3-amino-2-tert-butoxycarbonylamino-propionic acid (9.98 g, 48.9 mmol) and sodium bicarbonate (13.34 g, 158.8 mmol) in N,N-dimethylformamide (50 mL) was heated at 70° C. for 36 hours. The reaction was then cooled to room temperature, diluted with ethyl acetate (200 mL) and washed three times with 1:1 saturated aqueous NH₄Cl solution:H₂O. The aqueous wash layers were combined and extracted with ethyl acetate (50 mL). The ethyl acetate extracts were combined, washed with saturated aqueous NaCl solution (50 mL), dried over MgSO₄, filtered and concentrated in vacuo to give an oil that was used without further purification in the next reaction described below.

To a solution of the crude product described above in methanol (100 mL) was added 10% Pd/C (3.0 g). The reaction vessel was flushed with hydrogen, and the reaction stirred under an atmosphere of hydrogen for 4 days. The reaction mixture was then filtered through celite with the aid of ethyl acetate (200 mL). The resulting filtrate was concentrated in vacuo to give a solid that was used without further purification in the next reaction described below.

A mixture of the crude product described above, 143-(dimethylamino)propyl]-3-ethylcarbodiimide hydrochloride (12.3 g, 64.2 mmol) and N,N-dimethylformamide (150 mL) was stirred at room temperature for 4 hours. The reaction was then diluted with ethyl acetate (300 mL) and washed first with 1:1 saturated aqueous NaHCO₃ solution:H₂O (200 mL), then with H₂O (100 mL). The aqueous wash layers were combined and extracted with ethyl acetate (2×100 mL). The ethyl acetate extracts were combined, dried over MgSO₄, filtered and concentrated in vacuo to give an oil that was purified via chromatography on silica gel (20% to 40% ethyl acetate/hexanes linear gradient) to give the desired product.

¹H NMR (CDCl₃): δ 7.89 (s, 1H), 6.99 (m, 1H), 6.86 (d, J=6.8 Hz, 1H), 6.79 (dd, J=7.3, 7.3 Hz, 1H), 6.72 (d, J=8.0 Hz, 1H), 5.73 (d, J=5.3 Hz, 1H), 4.52 (m, 1H), 3.87 (dd, J=11.2, 3.8 Hz, 1H), 3.42 (dd, J=11.0, 11.0 Hz, 1H), 1.43 (s, 9H)⁺

MS: m/e 300.3 (M+23)⁺

### Step 2: Preparation of [(R)-2-oxo-1-(2,2,2-trifluoroethyl)-2,3,4,5-tetrahydro-1H-benzo[b][1,4]diazepin-3-yl)-carbamic acid tert-butyl ester

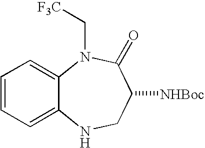

An oven-dried 100 mL round-bottom flask containing the product of Step 1 (1.98 g, 7.14 mmol) was fitted with a stirbar and septa and flushed with nitrogen. Tetrahydrofuran (20 mL) was added, giving a solution that was cooled to 0° C. A solution of potassium bis(trimethylsilyl)amide in toluene (0.5 M, 19 mL) was added, and the resulting mixture was stirred at 0° C. for 30 minutes. Trifluoromethanesulfonic acid-2,2,2-trifluoroethyl ester (1.54 g, 8.64 mmol) was then added, and the reaction was stirred with slow warming to room temperature. After 18 hours, additional trifluoromethanesulfonic acid-2,2,2-trifluoroethyl ester (0.70 g, 3.93 mmol) was added, and the reaction was stirred for 2 hours more. The reaction was then diluted with ethyl acetate (150 mL) and washed with 1:1 saturated aqueous NaHCO₃ solution:H₂O (2×50 mL). The aqueous wash layers were combined and extracted with ethyl acetate (50 mL). The ethyl acetate extracts were combined, washed with saturated aqueous NaCl solution (50 mL), dried over MgSO₄, filtered and concentrated in vacuo to give an oil that was purified via chromatography on silica gel (5% to 40% ethyl acetate/hexanes linear gradient) to give the desired product.

¹H NMR (CDCl₃): δ 7.14 (m, 2H), 7.04 (m, 1H), 6.90 (dd, J=7.7, 1.1 Hz, 1H), 5.48 (d, J=7.3 Hz, 1H), 4.79 (m, 1H), 4.62 (ddd, J=11.5, 7.1, 7.1 Hz, 1H), 4.14 (m, 1H), 3.89 (m, 1H), 3.38 (m, 2H), 1.40 (s, 9H)

MS: m/e 382.3 (M+23)⁺

### Step 3: Preparation of [(R)-5-cyclopropylmethyl-2-oxo-1-(2,2,2-trifluoroethyl)-2,3,4,5-tetrahydro-1H-benzo[b][1,4]diazepin-3-yl)-carbamic acid tert-butyl ester

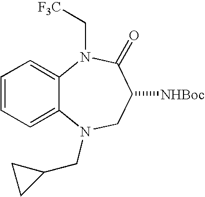

To a heavy-walled sealable pressure tube were added the product of Step 2 (1.20 g, 3.34 mmol), potassium carbonate (1.81 g, 13.0 mmol), tetrabutylammonium iodide (0.19 g, 0.51 mmol), tetrahydrofuran (10 mL) and bromomethyl cyclopropane (2.50 mL, 3.48 g, 25.7 mmol), in that order. The tube was sealed tightly with a teflon screwcap, and the reaction was heated at 100° C. for 18 hours. The reaction was then diluted with ethyl acetate (100 mL) and washed first with saturated aqueous NaHCO₃ solution (2×50 mL), then with saturated aqueous NaCl solution (50 mL). The organic layer was dried over MgSO₄, filtered and concentrated in vacuo to give an oil that was purified via chromatography on silica gel (0% to 35% ethyl acetate/hexanes linear gradient) to give the desired product.

¹H NMR (CDCl₃): δ 7.24 (m, 1H), 7.17 (d, J=7.8 Hz, 1H), 7.09 (m, 1H), 5.46 (d, J=7.5 Hz, 1H), 4.91 (m, 1H), 4.43 (ddd, J=11.5, 7.4, 7.4 Hz, 1H), 3.99 (m, 1H), 3.52 (dd, J=11.4, 9.4 Hz, 1H), 3.32 (dd, J=9.2, 7.1 Hz, 1H), 3.15 (dd, J=12.4, 5.3 Hz, 1H), 2.56 (dd, J=12.5, 7.7 Hz, 1H), 1.40 (s, 9H), 0.89 (m, 1H), 0.54 (dddd, J=9.3, 9.3, 4.8, 4.8 Hz, 1H), 0.46 (dddd, J=8.3, 8.3, 4.1, 4.1 Hz, 1H), 0.15 (m, 2H)

MS: m/e 436.3 (M+23)⁺

### Step 4: Preparation of [(R)-1-[(R)-5-cyclopropylmethyl-2-oxo-1-(2,2,2-trifluoro-ethyl)-2,3,4,5-tetrahydro-1H-benzo[b][1,4]diazepin-3-ylcarbamoyl]-2-(4-fluoro-phenyl)-ethyl]-carbamic acid tert-butyl ester

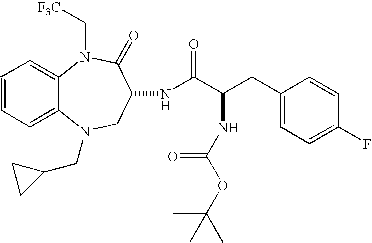

To a solution of the product of Step 3 (0.730 g, 1.77 mmol) in dichloromethane (8 mL) was added trifluoroacetic acid (2 mL). The resulting solution was stirred at room temperature for 1 hour, then concentrated in vacuo to give an oil that was used without further purification in the next reaction described below.

To a solution of the crude product described above (0.11 mmol) in dichloromethane (1 mL) were added diisopropylethylamine (0.18 mL, 1.0 mmol), N-Boc-D-4-fluorophenylalanine (0.066 g, 0.23 mmol) and benzotriazol-1-yloxytris(dimethylamino)phosphonium hexafluorophosphate (0.076 g, 0.18 mmol), in that order. The resulting solution was stirred at room temperature for 8 hours, and was then loaded directly onto a silica gel column and purified via flash chromatography (20% to 35% ethyl acetate/hexanes linear gradient) to give the desired product.

¹H NMR (CDCl₃): δ 7.28 (m, 1H), 7.18 (dd, J=7.7, 1.3 Hz, 1H), 7.11 (m, 4H), 6.97 (m, 2H), 6.85 (br s, 1H), 4.87 (m, 2H), 4.55 (m, 1H), 4.34 (m, 1H), 3.98 (m, 1H), 3.40 (m, 2H), 3.14 (dd, J=12.4, 5.3 Hz, 1H), 3.03 (m, 1H), 2.97 (dd, J=13.7, 6.2 Hz, 1H), 2.55 (dd, J=12.5, 7.7 Hz, 1H), 1.42 (s, 9H), 0.86 (m, 1H), 0.50 (m, 2H), 0.14 (m, 2H)

MS: m/e 601.3 (M+23)⁺

Examples listed below in TABLE 1 were prepared according to the procedures given above for the preparation of EXAMPLE 1 using the appropriate commercially available starting materials. For some examples, the required N-Boc protected D-phenylalanine derivative (as used in Step 4 above) was not commercially available. In those cases, the phenylalanine derivative was synthesized via the method of Scholkopf or Williams noted above in Scheme 1.

TABLE 1

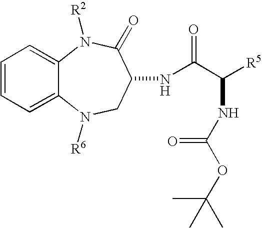

| Ex- | | | | |
|---|---|---|---|---|
| ample | | | | (m/e) |
| # | R² | R⁵ | R⁶ | (M + H) |
| 2 | H | 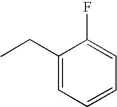 | H | 465.5 (M + Na) |
| 3 | Me | 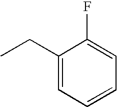 | H | 457.0 |
| 4 | Me | 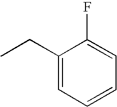 | Me | 471.0 |
| 5 | Me | 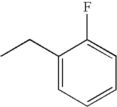 | 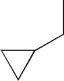 | 511.2 |
| 6 | Me | 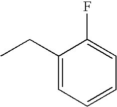 | 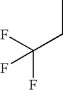 | 539.0 |
| 7 | Me | 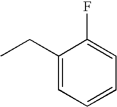 | 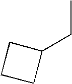 | 525.2 |
| 8 | Me | 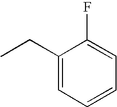 | 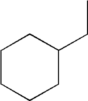 | 553.7 |
| 9 | Me | 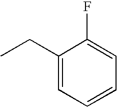 | 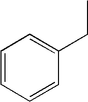 | 547.6 |
| 10 | i-Pr | 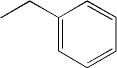 | H | 467.5 |
| 11 | i-Pr | 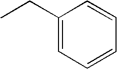 | 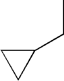 | 521.6 |
| 12 | i-Pr | 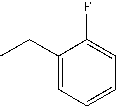 | 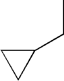 | 561.3 (M + Na) |
| 13 | i-Pr | 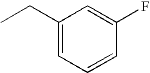 | 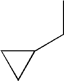 | 561.3 (M + Na) |
| 14 | i-Pr | 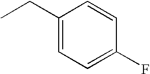 | 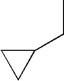 | 561.3 (M + Na) |
| 15 | i-Pr | 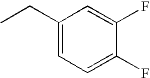 | 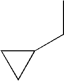 | 579.3 (M + Na) |
| 16 | i-Pr | 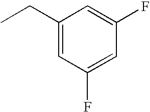 | 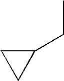 | 579.3 (M + Na) |
| 17 | i-Pr | 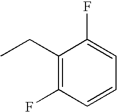 | 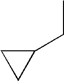 | 579.3 (M + Na) |
| 18 | i-Pr | 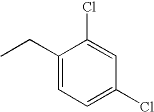 | 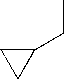 | 611.3 (M + Na) |
| 19 | i-Pr | 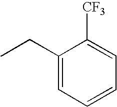 | 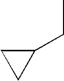 | 589.5 |
| 20 | i-Pr | 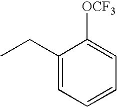 | 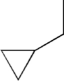 | 505.5 (M − Boc + H) |
| 21 | i-Pr | 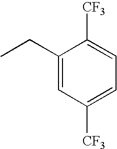 | 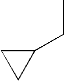 | 679.5 (M + Na) |
| 22 | 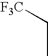 | 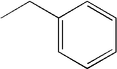 | 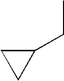 | 583.5 (M + Na) |
| 23 | 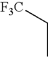 | 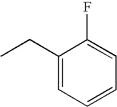 | 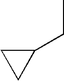 | 601.3 (M + Na) |
| 24 | 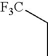 | 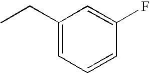 | 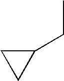 | 601.3 (M + Na) |
| 25 | 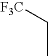 | 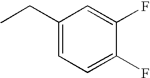 | 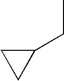 | 619.3 (M + Na) |
| 26 | 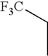 | 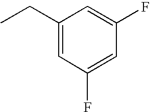 | 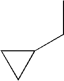 | 619.3 (M + Na) |
| 27 | 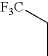 | 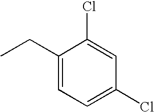 | 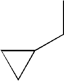 | 651.2 (M + Na) |
| 28 | 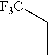 | 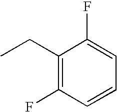 | 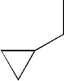 | 619.3 (M + Na) |
| 29 | 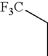 | 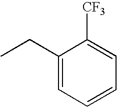 | 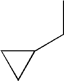 | 651.4 (M + Na) |
| 30 | 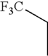 | 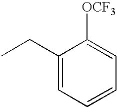 | 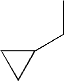 | 667.4 (M + Na) |
| 31 | 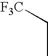 | 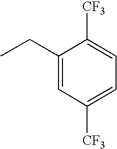 | 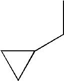 | 719.4 (M + Na) |

### Example 32

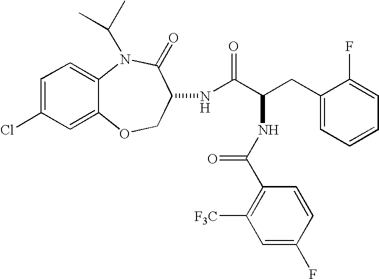

### N—[(R)-1-((R)-3-chloro-9-isopropyl-8-oxo-6,7,8,9-tetrahydro-5-oxa-9-aza-benzocyclohepten-7-ylcarbamoyl)-2-(2-fluoro-phenyl)-ethyl]-4-fluoro-2-trifluoromethyl-benzamide

### Step 1: Preparation of ((R)-3-chloro-8-oxo-6,7,8,9-tetrahydro-5-oxa-9-aza-benzocyclohepten-7-yl)-carbamic acid tert-butyl ester

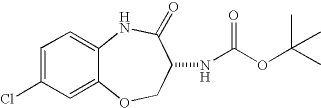

A solution of ((R)-8-oxo-6,7,8,9-tetrahydro-5-oxa-9-aza-benzocyclohepten-7-yl)-carbamic acid tert-butyl ester (5.0 g, 18 mmol, prepared as described previously: DeVita, R. J., Schoen, W. R., Doldouras, G. A., Fisher, M. H., Wyratt, M. J., Cheng, K., Chan, W. W.-S., Butler, B. S., Smith, R. G. Heterocyclic Analogs of the Benzolactam Nucleus of the Non-Peptidic Growth Hormone Secretagogue L-692,429. Bioorganic & Medicinal Chemistry Letters, 5, 1281-1286 (1995)) and N-chlorosuccinimide (3.12 g, 23.4 mmol) in N,N-dimethylformamide (30 mL) was stirred at room temperature for 5 hours. The reaction mixture was then diluted with dichloromethane and washed three times with H₂O and then once with saturated aqueous NaCl solution. The organic layer was separated, dried over MgSO₄, filtered and concentrated in vacuo to give an oil that was purified via flash chromatography on silica gel (17% ethyl acetate/hexanes) to give the desired product.

¹H NMR (CDCl₃): δ 7.79 (br s, 1H), 7.18 (d, J=2.5 Hz, 1H), 7.11 (dd, J=8.5, 2.5 Hz, 1H), 6.96 (d, J=8.5 Hz, 1H), 5.50 (br s, 1H), 4.67 (m, 2H), 4.25 (m, 1H), 1.46 (s, 9H)

MS: m/e 213.37 (M-Boc+1)⁺

### Step 2: Preparation of ((R)-3-chloro-9-isopropyl-8-oxo-6,7,8,9-tetrahydro-5-oxa-9-aza-benzocyclohepten-7-yl)-carbamic acid tert-butyl ester

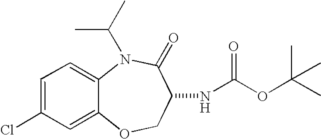

A suspension of sodium hydride (60%/oil, 0.052 g, 1.3 mmol) in N,N-dimethylformamide (3 mL) was cooled to 0° C. A solution of the product of Step 1 (0.312 g, 1.0 mmol) in N,N-dimethylformamide (3 mL) was then added, and the resulting mixture was allowed to warm to room temperature. After 30 minutes, the reaction was cooled to 0° C. 2-Iodopropane (0.340 g, 2.0 mmol) was added, and the reaction was allowed to warm to room temperature. After 1 hour, the reaction was poured into H₂O and extracted three times with dichloromethane. The organic extracts were combined, washed three times with H₂O and then once with saturated aqueous NaCl solution, dried over MgSO₄, filtered and concentrated in vacuo to give an oil that was purified via flash chromatography on silica gel (17% ethyl acetate/hexanes) to give the desired product.

¹H NMR (CDCl₃): δ 7.18 (m, 3H), 5.5 (br s, 1H), 4.78 (septet, J=6.5 Hz, 1H), 4.54 (m, 1H), 4.48 (m, 1H), 4.11 (m, 1H), 1.46 (d, J=6.5 Hz, 3H), 1.41 (s, 9H), 1.15 (d, J=6.5 Hz, 3H)

MS: m/e 255.40 (M-Boc+1)⁺

### Step 3: Preparation of [(R)-1-((R)-3-chloro-9-isopropyl-8-oxo-6,7,8,9-tetrahydro-5-oxa-9-aza-benzocyclohepten-7-ylcarbamoyl)-2-(2-fluoro-phenyl)-ethyl]-carbamic acid tert-butyl ester

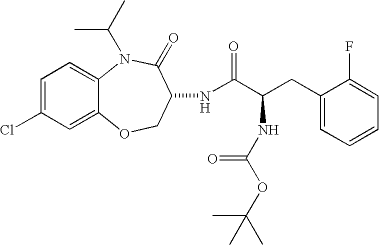

To the product of Step 2 (0.240 g, 0.678 mmol) was added a solution of HCl in methanol that had been prepared via the addition of acetyl chloride (2.0 mL, 28 mmol) to methanol (20 mL). The resulting reaction mixture was stirred at room temperature for 8 hours, then concentrated in vacuo to give a solid that was used without further purification in the next reaction described below.

To a mixture of the crude product described above (0.180 g) in dichloromethane (5 mL) were added N,N-diisopropylethylamine (0.320 g, 2.48 mmol), N-Boc-D-2-fluorophenylalanine (0.193 g, 0.680 mmol), 1-hydroxybenzotriazole (0.092 g, 0.68 mmol) and benzotriazol-1-yloxytris(dimethylamino)phosphonium hexafluorophosphate (0.302 g, 0.68 mmol). The resulting reaction mixture was stirred at room temperature for 1 hour, then diluted with dichloromethane and washed sequentially with H₂O and saturated aqueous NaCl solution. The organic layer was separated, dried over MgSO₄, filtered and concentrated in vacuo to give a residue that was purified via flash chromatography on silica gel (30% ethyl acetate/hexanes) to give the desired product.

¹H NMR (CDCl₃): δ 7.40-7.22 (m, 5H), 7.09-7.02 (m, 2H), 4.70 (m, 1H), 4.65 (septet, J=7.0 Hz, 1H), 4.41 (dd, J=10.0, 7.5 Hz, 1H), 4.36 (dd, J=9.5, 5.0 Hz, 1H), 4.19 (t, J=11.0 Hz, 1H), 3.19 (dd, J=14.0, 5.5 Hz, 1H), 2.85 (dd, J=14.0, 10.0 Hz, 1H), 1.14 (d, J=7.0 Hz, 3H), 1.35 (s, 9H), 1.16 (d, J=7.0 Hz, 3H)

MS: m/e 420.37 (M-Boc+1)⁺

### Step 4: Preparation of N—[(R)-1-((R)-3-chloro-9-isopropyl-8-oxo-6,7,8,9-tetrahydro-5-oxa-9-aza-benzocyclohepten-7-ylcarbamoyl)-2-(2-fluoro-phenyl)-ethyl]-4-fluoro-2-trifluoromethyl-benzamide

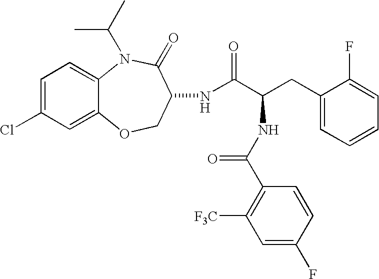

To the product of Step 3 (0.250 g, 0.48 mmol) was added a solution of HCl in methanol that had been prepared via the addition of acetyl chloride (2.0 mL, 28 mmol) to methanol (20 mL). The resulting reaction mixture was stirred at room temperature for 8 hours, then concentrated in vacuo to give a solid that was used without further purification in the next reaction described below.

To a mixture of the crude product described above (0.040 g) in dichloromethane (2 mL) were added N,N-diisopropylethylamine (0.045 g, 0.35 mmol), 4-fluoro-2-(trifluoromethyl)benzoic acid (0.018 g, 0.087 mmol), 1-hydroxybenzotriazole (0.012 g, 0.087 mmol) and benzotriazol-1-yloxytris(dimethylamino)phosphonium hexafluorophosphate (0.038 g, 0.087 mmol). The resulting reaction mixture was stirred at room temperature for 1 hour, then diluted with dichloromethane and washed sequentially with H₂O and saturated aqueous NaCl solution. The organic layer was separated, dried over MgSO₄, filtered and concentrated in vacuo to give a residue that was purified via flash chromatography on silica gel (5% methanol/dichloromethane) to give the desired product.

¹H NMR (CD₃OD): δ 7.69 (m, 1H), 7.61 (m, 2H), 7.40 (d, J=8.5 Hz, 1H), 7.34-7.25 (m, 5H), 7.20-7.05 (m, 2H), 4.91 (dd, J=9.5, 5.5 Hz, 1H), 4.74 (m 1H), 4.68 (septet, J=7.0 Hz, 1 H), 4.42 (dd, J=10.0, 7.5 Hz, 1H), 4.22 (dd, J=11.0, 9.5 Hz, 1H), 3.26 (m, 1H), 3.03 (m, 1H), 1.44 (d, J=7.0 Hz, 3H), 1.16 (d, J=7.0 Hz, 3H)

MS: m/e 529.09 (M+1)⁺

Examples listed below in TABLE 2 were prepared according to the procedures given above for the preparation of EXAMPLE 32 using the appropriate commercially available starting materials. For some examples, the required N-Boc protected D-phenylalanine derivative (as used in Step 3 above) was not commercially available. In those cases, the phenylalanine derivative was synthesized via the method of Scholkopf or Williams noted above in Scheme 1.

TABLE 2

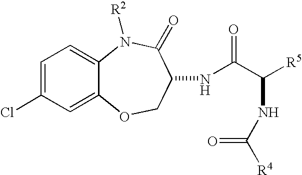

| Example | | | | (m/e) |
|---|---|---|---|---|
| # | R² | R⁴ | R⁵ | (M + H) |
| 33 | 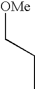 | OC(CH₃)₃ | 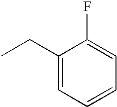 | 436.0 (M − Boc + H) |
| 34 | 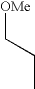 | OC(CH₃)₃ | 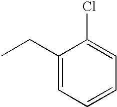 | 452.0 (M − Boc + H) |
| 35 | 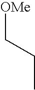 | 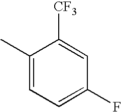 | 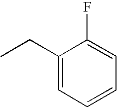 | 648.1 (M + Na) |
| 36 | 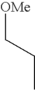 | 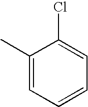 | 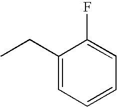 | 596.1 (M + Na) |
| 37 | 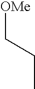 | 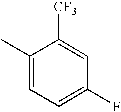 | 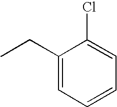 | 664.1 (M + Na) |
| 38 | 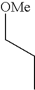 | 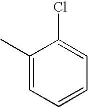 | 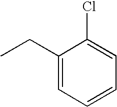 | 613.9 (M + Na) |
| 39 | i-Pr | OC(CH₃)₃ | 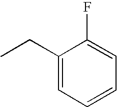 | 420.64 (M − Boc + H) |
| 40 | i-Pr | OC(CH₃)₃ | 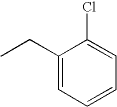 | 436.6 (M − Boc + H) |
| 41 | i-Pr | OC(CH₃)₃ | 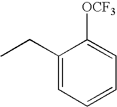 | 486.0 (M − Boc + H) |
| 42 | i-Pr | 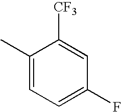 | 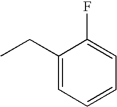 | 610.7 |
| 43 | i-Pr | OC(CH₃)₃ | 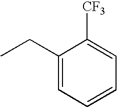 | 470.0 (M − Boc + H) |
| 44 | i-Pr | 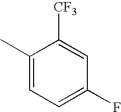 | 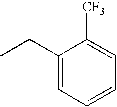 | 660.6 |
| 45 | 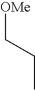 | OC(CH₃)₃ | 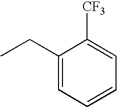 | 486.1 (M − Boc + H) |
| 46 | 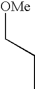 | OC(CH₃)₃ | 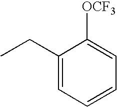 | 502.1 (M − Boc + H) |
| 47 | 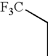 | OC(CH₃)₃ | 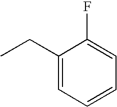 | 582.1 (M + Na) |
| 48 | 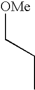 | 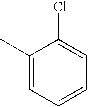 | 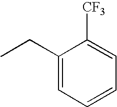 | 646.07 (M + Na) |
| 49 | 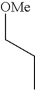 | 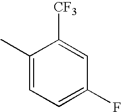 | 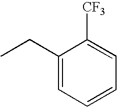 | 698.1 (M + Na) |
| 50 | 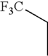 | 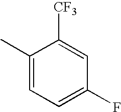 | 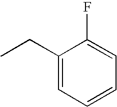 | 672.1 (M + Na) |
| 51 | 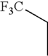 | 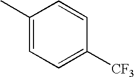 | 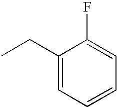 | 668.2 (M + Na) |
| 52 | i-Pr | 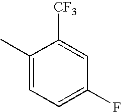 | 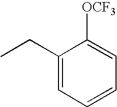 | 676.6 (M + H) |
| 53 | 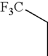 | 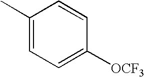 | 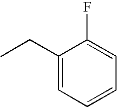 | 684.2 (M + Na) |
| 54 | 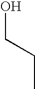 | OC(CH₃)₃ | 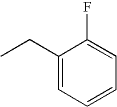 | 422.1 (M − Boc + H) |
| 55 | 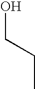 | OC(CH₃)₃ | 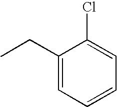 | 438.0 (M − Boc + H) |
| 56 | i-Pr | 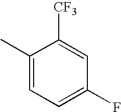 | 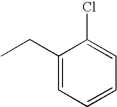 | 626.6 |
| 57 | 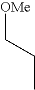 | 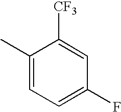 | 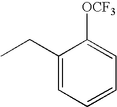 | 714.1 (M + Na) |
| 58 | 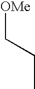 | 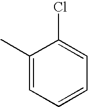 | 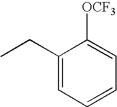 | 662.1 (M + Na) |
| 59 | 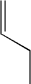 | OC(CH₃)₃ | 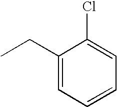 | 434.0 (M − Boc + H) |
| 60 | 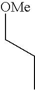 | OC(CH₃)₃ | 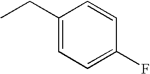 | 558.1 (M + Na) |
| 61 | 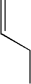 | 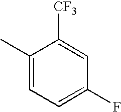 | 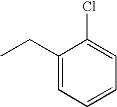 | 646.0 (M + Na) |
| 62 | 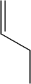 | 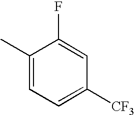 | 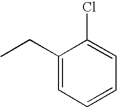 | 646.0 (M + Na) |
| 63 | 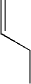 | 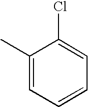 | 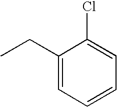 | 595.9 (M + Na) |
| 64 | 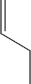 | 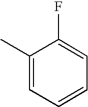 | 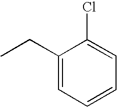 | 578.0 (M + Na) |
| 65 | 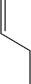 | 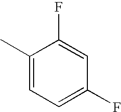 | 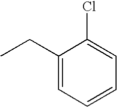 | 596.0 (M + Na) |
| 66 | i-Pr | OC(CH₃)₃ | 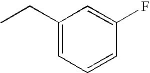 | 420.6 (M − Boc + H) |
| 67 | i-Pr | 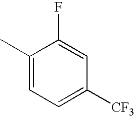 | 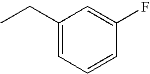 | 610.0 |
| 68 | 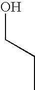 | OC(CH₃)₃ | 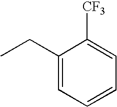 | 471.9 (M − Boc + H) |
| 69 | i-Pr | 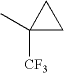 | 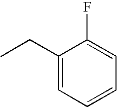 | 556.6 |
| 70 | i-Pr | 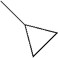 | 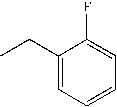 | 488.1 |
| 71 | i-Pr | 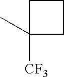 | 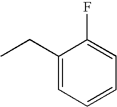 | 570.7 |
| 72 | i-Pr | 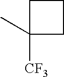 | 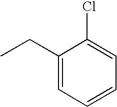 | 586.6 |
| 73 | i-Pr | 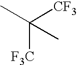 | 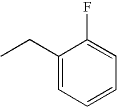 | 612.1 |
| 74 | i-Pr | 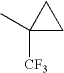 | 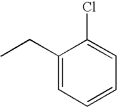 | 572.6 |
| 75 | i-Pr | 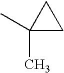 | 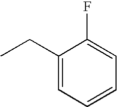 | 502.6 |
| 76 | i-Pr | 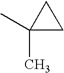 | 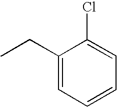 | 518.6 |
| 77 | i-Pr | 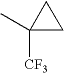 | 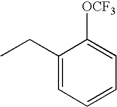 | 622.1 |
| 78 | i-Pr | 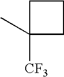 | 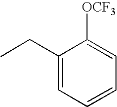 | 636.5 |
| 79 | i-Pr | 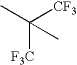 | 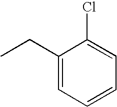 | 628.5 |
| 80 | i-Pr | 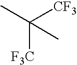 | 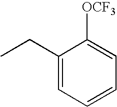 | 678.2 |
| 81 | i-Pr | 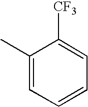 | 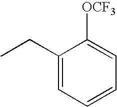 | 658.6 |
| 82 | i-Pr | 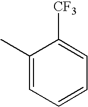 | 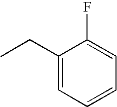 | 592.2 |
| 83 | i-Pr | 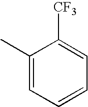 | 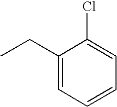 | 608.4 |
| 84 | i-Pr | t-Bu | 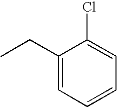 | 520.8 |
| 85 | i-Pr | t-Bu | 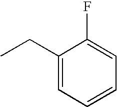 | 504.8 |
| 86 | i-Pr | 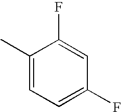 | 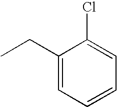 | 576.2 |
| 87 | i-Pr | 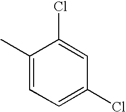 | 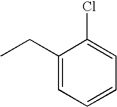 | 610.2 |
| 88 | i-Pr | 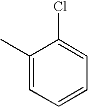 | 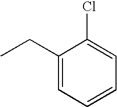 | 576.2 |
| 89 | i-Pr | 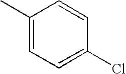 | 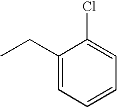 | 576.6 |

### Example 90

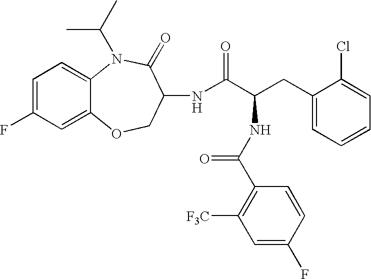

### N—[(R)-2-(2-Chloro-phenyl)-1-(3-fluoro-9-isopropyl-8-oxo-6,7,8,9-tetrahydro-5-oxa-9-aza-benzocyclohepten-7-ylcarbamoyl)-ethyl]-4-fluoro-2-trifluoromethyl-benzamide

### Step 1: Preparation of 2-[3-(5-fluoro-2-nitro-phenoxy)-propoxy]-tetrahydro-pyran

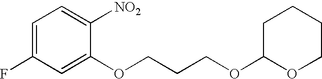

A mixture of 5-fluoro-2-nitrophenol (16.75 g, 106.7 mmol) and potassium hydroxide (8.97 g, 160 mmol) in N,N-dimethylformamide (250 mL) was heated at 40° C. for 2 hours. 2-(3-bromopropoxy)tetrahydro-2H-pyran (23.8 g, 106.7 mmol) was added, and the resulting mixture was heated at 60° C. for 4 hours, then cooled to room temperature. The reaction was then diluted with H₂O and extracted with ethyl acetate. The organic extracts were combined, washed sequentially with H₂O (3×300 mL) and saturated aqueous NaCl solution, dried over MgSO₄, filtered and concentrated in vacuo to give an oil that was purified via flash chromatography on silica gel (10% ethyl acetate/hexanes) to give the desired product.

¹H NMR (CDCl₃): δ 7.96 (dd, J=9.0, 6.0 Hz, 1H), 6.84 (dd, J=10.5, 2.5 Hz, 1H), 6.73 (m, 1H), 4.60 (dd, J=4.5, 2.5 Hz, 1H), 4.25 (m, 2H), 3.97 (m, 1H), 3.86 (m, 1H), 3.65 (m, 1H), 3.52 (m, 1H), 2.21 (m, 2H), 1.83 (m, 1H), 1.74 (m, 1H), 1.57 (m, 4H)

MS: m/e 322.16 (M+23)⁺

### Step 2: Preparation of 3-(5-fluoro-2-nitro-phenoxy)-propan-1-ol

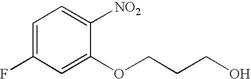

A solution of the product of Step 1 (18.42 g, 61.58 mmol) in acetic acid (100 mL) and H₂O (20 mL) was heated at 50° C. for 6 hours. The reaction was then cooled to room temperature, diluted with H₂O and extracted with ethyl acetate. The organic extracts were combined, washed sequentially with H₂O and saturated aqueous NaCl solution, dried over MgSO₄, filtered and concentrated in vacuo to give an oil that was purified via flash chromatography on silica gel (30% ethyl acetate/hexanes) to give the desired product.

¹H NMR (CDCl₃): δ 8.02 (dd, J=9.0, 5.5 Hz, 1H), 6.84 (dd, J=10.5, 2.5 Hz, 1H), 6.76 (m, 1H), 4.29 (t, J=5.5 Hz, 2H), 3.94 (t, J=5.5 Hz, 1H), 2.15 (quintet, J=5.5 Hz, 2H)

MS: m/e 215.99 (M+1)⁺

### Step 3: Preparation of 3-fluoro-6,7-dihydro-9H-5-oxa-9-aza-benzocyclohepten-8-one

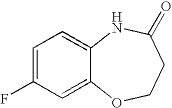

To a solution of the product of Step 2 (9.50 g, 44.2 mmol) in acetone (200 mL) was added Jones reagent (excess) in a dropwise manner over 1 hour. After 5 hours, 2-propanol (10 mL) was added to quench any excess reagent. The reaction mixture was stirred for 1 hour, then filtered and concentrated in vacuo. The resulting residue was diluted with ethyl acetate, washed sequentially with H₂O and saturated aqueous NaCl solution, dried over MgSO₄, filtered and concentrated in vacuo to give a solid that was used without further purification in the next reaction described below.

A mixture of the crude product described above (8.51 g) and 10% Pd/C (0.400 g) in methanol (50 mL) was shaken under hydrogen (40 psi) for 5 hours. The reaction mixture was then filtered and concentrated in vacuo to give a solid that was used without further purification in the next reaction described below.

To a cooled 0° C. mixture of the crude product described above (7.4 g) in dichloromethane (100 mL) were added N,N-diisopropylethylamine (26.0 mL, 149 mmol), 1-hydroxybenzotriazole (5.52 g, 40.8 mmol) and benzotriazol-1-yloxytris(dimethylamino)phosphonium hexafluorophosphate (18.1 g, 40.8 mmol). The resulting reaction mixture was stirred at room temperature for 3 hours, then diluted with dichloromethane and washed sequentially with H₂O and saturated aqueous NaCl solution. The organic layer was separated, dried over MgSO₄, filtered and concentrated in vacuo to give a residue that was purified via flash chromatography on silica gel (25% ethyl acetate/hexanes) to give the desired product.

¹H NMR (CDCl₃): δ 6.91 (m, 1H), 6.81 (m, 2H), 4.53 (t, J=5.5 Hz, 2H), 2.86 (t, J=5.5 Hz, 2H)

MS: m/e 181.96 (M+1)⁺

### Step 4: Preparation of 3-fluoro-9-isopropyl-6,7-dihydro-9H-5-oxa-9-aza-benzocyclohepten-8-one

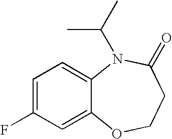

To a cooled 0° C. mixture of sodium hydride (60%/oil, 0.159 g, 3.98 mmol) in N,N-dimethylformamide (5 mL) was added a solution of the product of Step 3 (0.600 g, 3.30 mmol) in N,N-dimethylformamide (5 mL). The resulting mixture was allowed to warm to room temperature over 30 minutes, and was then cooled back to 0° C. 2-Iodopropane (0.732 g, 4.30 mmol) was added, and the reaction was stirred for 2 hours while slowly warming to room temperature, then diluted with H₂O and extracted with ethyl acetate. The organic extracts were combined, washed sequentially with H₂O and saturated aqueous NaCl solution, dried over MgSO₄, filtered and concentrated in vacuo to give a residue that was purified via flash chromatography on silica gel (20% ethyl acetate/hexanes) to give the desired product.

¹H NMR (CDCl₃): δ 7.22 (dd, J=8.5, 6.0 Hz, 1H), 6.91 (m, 2H), 4.79 (septet, J=7.0 Hz, 1H), 4.54 (m, 2H), 2.58 (m, 2H), 1.28 (d, J=7.0 Hz, 6H)

MS: m/e 224.03 (M+1)⁺

### Step 5: Preparation of 3-fluoro-7-iodo-9-isopropyl-6,7-dihydro-9H-5-oxa-9-aza-benzocyclohepten-8-one

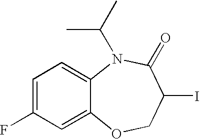

A solution of the product of Step 4 (0.300 g, 1.34 mmol) in dichloromethane (6 mL) was cooled to −10° C. N,N,N′,N′-Tetramethylethlenediamine (1.02 mL, 6.72 mmol) and iodotrimethylsilane (1.35 g, 6.72 mmol) were added, in that order, giving a mixture that was stirred for 30 minutes. Iodine (1.03 g, 4.03 mmol) was then added, and the reaction was allowed to slowly warm to room temperature. After 1 hour, the reaction mixture was diluted with H₂O and extracted with ethyl acetate. The organic extracts were combined, washed sequentially with saturated aqueous NaHSO₃ solution and saturated aqueous NaCl solution, dried over MgSO₄, filtered and concentrated in vacuo to give a residue that was purified via flash chromatography on silica gel (10% ethyl acetate/hexanes) to give the desired product.

¹H NMR (CDCl₃): δ 7.28 (m, 1H), 6.90 (m, 2H), 4.83 (m, 1H), 4.75 (m, 2H), 4.70 (septet, J=7.0 Hz, 1H), 4.50 (m, 1H), 1.41 (d, J=7.0 Hz, 3H), 1.20 (d, J=7.0 Hz, 3H)

### Step 6: Preparation of 7-amino-3-fluoro-9-isopropyl-6,7-dihydro-9H-5-oxa-9-aza-benzocyclohepten-8-one

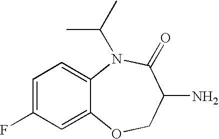

A mixture of the product of Step 5 (0.240 g, 0.690 mmol) and sodium azide (0.178 g, 2.75 mmol) in N,N-dimethylformamide (6 mL) was heated at 40° C. for 5 hours. The reaction was then cooled to room temperature, diluted with ethyl acetate, washed sequentially with H₂O and saturated aqueous NaCl solution, dried over MgSO₄, filtered and concentrated in vacuo to give a residue that was used without further purification in the next reaction described below.

A mixture of the crude product described above (0.175 g) and 10% Pd/C (0.050 g) in methanol (10 mL) was stirred at room temperature under an atmosphere of hydrogen. After 18 hours, the reaction was filtered and concentrated in vacuo to give the desired product.

¹H NMR (CDCl₃): δ 7.20 (m, 1H), 6.95 (m, 2H), 4.80 (septet, J=7.0 Hz, 1H), 4.35 (m, 1H), 4.10 (m, 1H), 3.70 (m, 1H), 1.41 (d, J=7.0 Hz, 3H), 1.17 (d, J=7.0 Hz, 3H)

MS: m/e 239.36 (M+1)⁺

### Step 7: Preparation of [(R)-2-(2-chloro-phenyl)-1-(3-fluoro-9-isopropyl-8-oxo-6,7,8,9-tetrahydro-5-oxa-9-aza-benzocyclohepten-7-ylcarbamoyl)-ethyl]-carbamic acid tert-butyl ester

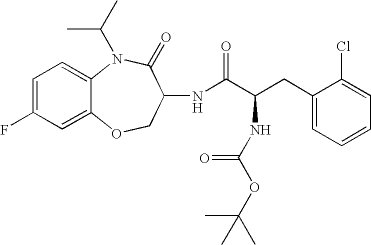

To a solution of the product of Step 6 (0.100 g, 0.42 mmol) in dichloromethane (6 mL) were added N,N-diisopropylethylamine (0.29 mL, 1.68 mmol), N-Boc-D-2-chlorophenylalanine (0.138 g, 0.46 mmol), 1-hydroxybenzotriazole (0.063 g, 0.46 mmol) and benzotriazol-1-yloxytris(dimethylamino)phosphonium hexafluorophosphate (0.204 g, 0.46 mmol). The resulting reaction mixture was stirred at room temperature for 1 hour, then diluted with ethyl acetate and washed sequentially with H₂O and saturated aqueous NaCl solution. The organic layer was separated, dried over MgSO₄, filtered and concentrated in vacuo to give a residue that was purified via high-pressure liquid chromatography (ChiralPak AD column, 20% 2-propanol/heptane) to give the product as a faster-eluting diastereomer (d1) and a slower-eluting diastereomer (d2).

d1 ¹H NMR (CD₃OD): δ 7.42 (dd, J=8.5, 5.5 Hz, 1H), 7.37 (m, 1H), 7.27 (m, 1H), 7.21 (m, 2H), 7.07 (m, 2H), 4.72 (m, 1H), 4.67 (septet, J=7 Hz, 1H), 4.43 (dd, J=10.0, 7.5 Hz, 2H), 4.19 (t, J=10.5 Hz, 1H), 3.30 (dd, J=14.0, 5.0 Hz, 1H), 2.94 (dd, J=14.0, 9.5 Hz, 1H), 1.44 (d, J=7 Hz, 3H), 1.36 (s, 9H), 1.14 (d, J=7 Hz, 3H)

d1 MS: m/e 420.19 (M-Boc+1)⁺

d2 ¹H NMR (CD₃OD): δ 7.42 (dd, J=8.5, 5.5 Hz, 1H), 7.38 (m, 1H), 7.25 (m, 3H), 7.06 (m, 2H), 4.69 (m, 2H), 4.45 (dd, J=9.0, 6.0 Hz, 1H), 4.32 (m, 1H), 4.07 (t, J=10.0 Hz, 1H), 3.28 (dd, J=13.5, 5.5 Hz, 1H), 2.98 (dd, J=9.0, 5.0 Hz, 1H), 1.44 (d, J=7 Hz, 3H), 1.36 (s, 9H), 1.13 (d, J=7 Hz, 3H)

d2 MS: m/e 420.64 (M-Boc+1)⁺

### Step 8: Preparation of N—[(R)-2-(2-chloro-phenyl)-1-(3-fluoro-9-isopropyl-8-oxo-6,7,8,9-tetrahydro-5-oxa-9-aza-benzocyclohepten-7-ylcarbamoyl)-ethyl]-4-fluoro-2-trifluoromethyl-benzamide

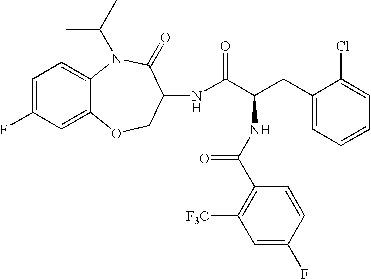

The faster-eluting product d1 of Step 7 (0.24 g, 0.68 mmol) was treated with a solution of hydrochloric acid in methanol. The resulting mixture was stirred at room temperature for 18 hours, then concentrated in vacuo to give a solid that was used without further purification in the next reaction described below.

To a mixture of the crude product described above (0.091 g) in dichloromethane (3 mL) were added N,N-diisopropylethylamine (0.16 g, 1.24 mmol), 4-fluoro-2-(trifluoromethyl)benzoic acid (0.066 g, 0.22 mmol), 1-hydroxybenzotriazole (0.031 g, 0.23 mmol) and benzotriazol-1-yloxytris(dimethylamino)phosphonium hexafluorophosphate (0.102 g, 0.23 mmol). The resulting reaction mixture was stirred at room temperature for 1 hour, then diluted with dichloromethane and washed sequentially with H₂O and saturated aqueous NaCl solution. The organic layer was separated, dried over MgSO₄, filtered and concentrated in vacuo to give a residue that was purified via flash chromatography on silica gel (10% methanol/dichloromethane) to give the desired product.

¹H NMR (CD₃OD): δ7.71 (m, 1H), 7.61 (m, 2H), 7.41 (m, 2H), 7.33 (m, 2H), 7.25 (m, 2H), 7.06 (m, 2H), 4.97 (dd, J=9.5, 6.0 Hz, 1H), 4.71 (m, 2H), 4.43 (dd, J=9.5, 7.0 Hz, 1H), 4.20 (dd, J=11.5, 10.0 Hz, 1H), 3.34 (dd, J=14.5, 8.5 Hz, 1H), 3.13 (dd, J=14.5, 8.5 Hz, 1H), 1.43 (d, J=7.0 Hz, 3H), 1.13 (d, J=7.0 Hz, 3H)

MS: m/e 592.65 (M+1)⁺

Examples listed below in TABLE 3 were prepared according to the procedures given above for the preparation of EXAMPLE 90 using the appropriate commercially available starting materials. The carbon atom marked with a * has the stereochemical configuration (R or S) listed for each example.

TABLE 3

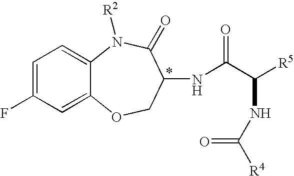

| Example | | | | | (m/e) |
|---|---|---|---|---|---|
| # | * | R² | R⁴ | R⁵ | (M + H) |
| 91 | R | i-Pr | OC(CH₃)₃ | 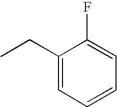 | 404.3 (M − Boc + H) |
| 92 | S | i-Pr | OC(CH₃)₃ | 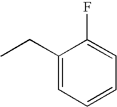 | 404.3 (M − Boc + H) |
| 93 | R | i-Pr | 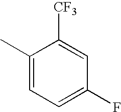 | 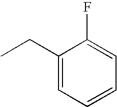 | 594.2 |
| 94 | R | i-Pr | 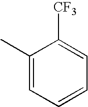 | 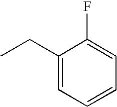 | 576.6 |
| 95 | S | i-Pr | 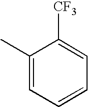 | 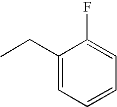 | 576.4 |
| 96 | R | i-Pr | 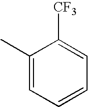 | 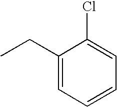 | 592.7 |
| 97 | S | i-Pr | 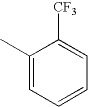 | 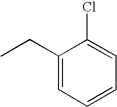 | 592.7 |
| 98 | R | i-Pr | OC(CH₃)₃ | 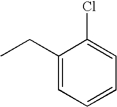 | 420.2 (M − Boc + H) |
| 99 | S | i-Pr | OC(CH₃)₃ | 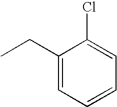 | 420.6 (M − Boc + H) |
| 100 | R | i-Pr | 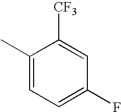 | 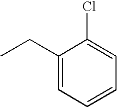 | 610.3 |
| 101 | S | i-Pr | 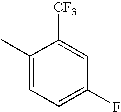 | 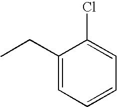 | 610.6 |
| 102 | R | i-Pr | 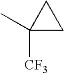 | 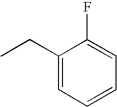 | 540.6 |
| 103 | R | i-Pr | t-Bu | 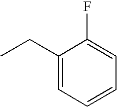 | 488.8 |
| 104 | S | i-Pr | t-Bu | 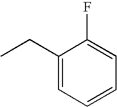 | 488.8 |
| 105 | R | i-Pr | OC(CH₃)₃ | 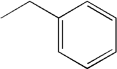 | 386.1 (M − Boc + H) |
| 106 | S | i-Pr | OC(CH₃)₃ | 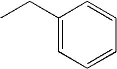 | 386.1 (M − Boc + H) |
| 107 | R | i-Pr | 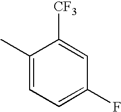 | 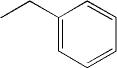 | 576.2 |
| 108 | R | i-Pr | 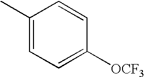 | 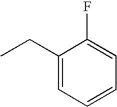 | 606.2 |
| 109 | R | i-Pr | 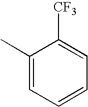 | 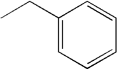 | 558.4 |
| 110 | S | i-Pr | 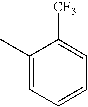 | 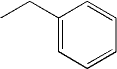 | 558.2 |
| 111 | R | i-Pr | 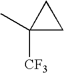 | 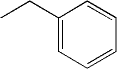 | 522.5 |
| 112 | R | i-Pr | 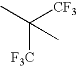 | 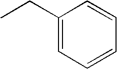 | 578.2 |
| 113 | R | i-Pr | 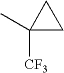 | 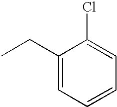 | 578.3 (M + Na) |
| 114 | S | i-Pr | 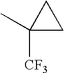 | 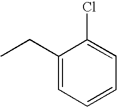 | 556.3 |

### Example 115

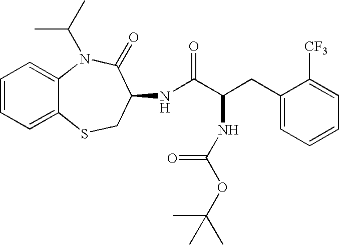

### [(R)-1-((R)-5-Isopropyl-4-oxo-2,3,4,5-tetrahydro-benzo[b][1,4]thiazepin-3-ylcarbamoyl)-2-(2-trifluoromethyl-phenyl)-ethyl]-carbamic acid tert-butyl ester

### Step 1: Preparation of ((R)-4-Oxo-2,3,4,5-tetrahydro-benzo[b][1,4]thiazepin-3-yl)-carbamic acid tert-butyl ester

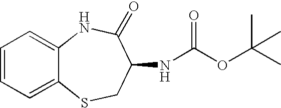

A solution of hydrogen bromide in acetic acid (15 mL) was added to ((R)-4-oxo-2,3,4,5-tetrahydro-benzo[b][1,4]thiazepin-3-yl)-carbamic acid benzyl ester (3.10 g, 9.45 mmol), which itself was prepared according to known procedures [Slade, J., Stanton, J. L., Ben-David, D., Mazzenga, G. C. Angiotensin Converting Enzyme Inhibitors: 1,5-Benzothiazepine Derivatives. Journal of Medicinal Chemistry 28, 1517-1521 (1985)]. The reaction was stirred at room temperature for 2 hours, then diluted with diethyl ether (200 mL) and stirred for 10 minutes. The resulting mixture was filtered and the solids collected to give a crude product which was used in the next reaction described below.

To a mixture of the crude product described above (0.825 g) in dichloromethane (20 mL) were added di-tert-butyl dicarbonate (0.677 g, 3.10 mmol) and triethylamine (0.62 mL, 4.0 mmol). The resulting reaction mixture was stirred overnight at room temperature, then poured into H₂O and extracted three times with dichloromethane. The organic extracts were combined, washed sequentially with H₂O and then aqueous NaCl solution, dried over MgSO₄, filtered and concentrated in vacuo to give a residue that was purified via chromatography on silica gel (30% to 60% ethyl acetate/hexanes linear gradient) to give the desired product.

¹H NMR (CDCl₃): δ 8.01 (s, 1H), 7.65 (d, J=7.7 Hz, 1H), 7.39 (dd, J=7.7, 7.6 Hz, 1H), 7.21 (dd, J=7.7, 7.6 Hz, 1H), 7.16 (d, J=7.6 Hz, 1H), 5.61 (br s, 1H), 4.50 (m, 1H), 3.83 (dd, J=11.1, 7.0 Hz, 1H), 2.99 (dd, J=11.2, 11.1 Hz, 1H), 1.41 (s, 9H)

MS: m/e 317.3 (M+23)⁺

### Step 2: Preparation of ((R)-5-Isopropyl-4-oxo-2,3,4,5-tetrahydro-benzo[b][1,4]thiazepin-3-yl)-carbamic acid tert-butyl ester

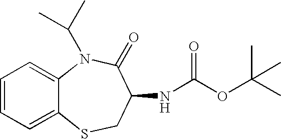

Sodium hydride (60% in oil, 0.026 g, 0.65 mmol) was added to a solution of the product of Step 1 (0.180 g, 0.542 mmol) in dimethylformamide (5 mL), and the resulting mixture was stirred at room temperature for 10 minutes. 2-Iodopropane (0.170 g, 1.0 mmol) was then added, and stirring was continued for 6 hours. The reaction mixture was then diluted with H₂O and extracted three times with ethyl acetate. The organic extracts were combined, washed sequentially with H₂O and saturated aqueous NaCl solution, dried over MgSO₄, filtered and concentrated in vacuo to give a residue that was purified via chromatography on silica gel (20% to 50% ethyl acetate/hexanes linear gradient) to give the desired product.

¹H NMR (CDCl₃): δ 7.64 (d, J=7.6 Hz, 1H), 7.41 (dd, J=7.6, 7.4 Hz, 1H), 7.28 (m, 2H), 5.61 (s, 1H), 4.91 (septet, J=7.1 Hz, 1H), 4.22 (m, 1H), 3.67 (dd, J=11.2, 7.0 Hz, 1H), 2.80 (t, J=11.2 Hz, 1H), 1.49 (d, J=7.1 Hz, 3H), 1.41 (s, 9H), 1.09 (d, J=7.0 Hz, 3H)

MS: m/e 237.3 (M-Boc+1)⁺

### Step 3: Preparation of [(R)-1-((R)-5-Isopropyl-4-oxo-2,3,4,5-tetrahydro-benzo[b][1,4]thiazepin-3-ylcarbamoyl)-2-(2-trifluoromethyl-phenyl)-ethyl]-carbamic acid tert-butyl ester

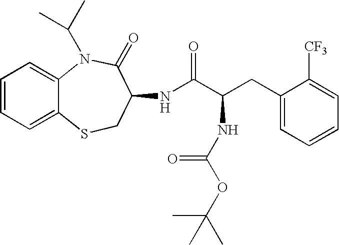

To the product of Step 2 (0.125 g, 0.372 mmol) was added a solution of hydrogen chloride in methanol (5 mL). The resulting solution was stirred at room temperature for 6 hours, then concentrated in vacuo to give a solid which was used without further purification in the next reaction described below.

To a mixture of 1-[3-(dimethylamino)propyl]-3-ethylcarbodiimide hydrochloride (0.095 g, 0.50 mmol), 1-hydroxybenzotriazole (0.068 g, 0.50 mmol) and N-Boc-D-2-trifluoromethyl phenylalanine (0.141 g, 0.399 mmol) in dichloromethane (5 mL) were added the crude product described above and diisopropylethylamine (0.18 mL, 1.0 mmol). The resulting solution was stirred at room temperature for 14 hours, then diluted with H₂O and extracted three times with dichloromethane. The organic extracts were combined, washed with saturated aqueous NaCl solution, dried over MgSO₄, filtered and concentrated in vacuo to give a residue that was purified via preparative thin-layer chromatography on silica gel (40% ethyl acetate/hexanes) to give the desired product.

¹H NMR (CDCl₃): δ 7.68 (m, 2H), 7.52 (m, 1H), 7.46 (m, 1H), 7.40 (m, 2H), 7.28 (m, 2H), 6.96 (s, 1H), 4.98 (s, 1H), 4.87 (septet, J=7.1 Hz, 1H), 4.49 (m, 1H), 3.71 (m, 1H), 3.37 (dd, J=7.8, 5.6 Hz, 1H), 3.02 (t, J=5.6 Hz, 1H), 2.63 (t, J=11.2 Hz, 1H), 1.49 (d, J=7.1 Hz, 3H), 1.41 (s, 9H), 1.09 (d, J=7.0 Hz, 3H)

MS: m/e 452.3 (M-Boc+1)⁺

Examples listed below in TABLE 4 were prepared according to the procedures given above for the preparation of EXAMPLE 115 using the appropriate commercially available starting materials. The carbon atom marked with a * has the stereochemical configuration (R or S) listed for each example.

TABLE 4

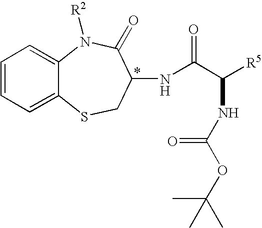

| | | | | (m/e) |
|---|---|---|---|---|
| Example # | * | R² | R⁵ | (M + H) |
| 116 | R | H | 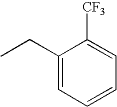 | 410.3 (M − Boc + H) |
| 117 | R | 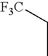 | 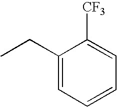 | 492.3 (M − Boc + H) |
| 118 | R | 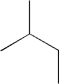 | 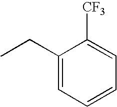 | 466.4 (M − Boc + H) |
| 119 | S | H | 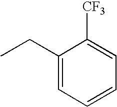 | 410.4 (M − Boc + H) |
| 120 | S | 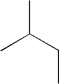 | 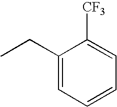 | 466.4 (M − Boc + H) |
| 121 | S | i-Pr | 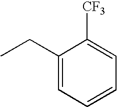 | 452.2 (M − Boc + H) |

The following in vitro and in vivo assays were used in assessing the biological activity of the instant compounds.

### Compound Evaluation (In Vitro Assay):

The identification of inhibitors of the sodium channel is based on the ability of sodium channels to cause cell depolarization when sodium ions permeate through agonist-modified channels. In the absence of inhibitors, exposure of an agonist-modified channel to sodium ions will cause cell depolarization. Sodium channel inhibitors will prevent cell depolarization caused by sodium ion movement through agonist-modified sodium channels. Changes in membrane potential can be determined with voltage-sensitive fluorescence resonance energy transfer (FRET) dye pairs that use two components, a donor coumarin (CC₂-DMPE) and an acceptor oxanol (DiSBAC₂(3)). Oxanol is a lipophilic anion and distributes across the membrane according to membrane potential. In the presence of a sodium channel agonist, but in the absence of sodium, the inside of the cell is negative with respect to the outside, oxanol is accumulated at the outer leaflet of the membrane and excitation of coumarin will cause FRET to occur. Addition of sodium will cause membrane depolarization leading to redistribution of oxanol to the inside of the cell, and, as a consequence, to a decrease in FRET. Thus, the ratio change (donor/acceptor) increases after membrane depolarization. In the presence of a sodium channel inhibitor, cell depolarization will not occur, and therefore the distribution of oxanol and FRET will remain unchanged.

Cells stably transfected with the PN1 sodium channel (HEK-PN1) were grown in polylysine-coated 96-well plates at a density of ca. 140,000 cells/well. The media was aspirated, and the cells were washed with PBS buffer, and incubated with 100 μL of 10 μm CC₂-DMPE in 0.02% pluronic acid. After incubation at 25° C. for 45 min, media was removed and cells were washed 2× with buffer. Cells were incubated with 100 μL it of DiSBAC₂(3) in TMA buffer containing 20 μM veratridine, 20 nM brevetoxin-3, and test sample. After incubation at 25° C. for 45 μM in the dark, plates were placed in the VIPR instrument, and the fluorescence emission of both CC₂-DMPE and DiSBAC₂(3) recorded for 10 s. At this point, 100 μL of saline buffer was added to the wells to determine the extent of sodium-dependent cell depolarization, and the fluorescence emission of both dyes recorded for an additional 20 s. The ratio CC₂-DMPE/DiSBAC₂(3), before addition of saline buffer equals 1. In the absence of inhibitors, the ratio after addition of saline buffer is >1.5. When the sodium channel has been completely inhibited by either a known standard or test compound, this ratio remains at 1. It is possible, therefore, to titrate the activity of a sodium channel inhibitor by monitoring the concentration-dependent change in fluorescence ratio.

### Electrophysiological Assays (In Vitro Assays):

Cell preparation: A HEK-293 cell line stably expressing the PN1 sodium channel subtype was established in-house. The cells were cultured in MEM growth media (Gibco) with 0.5 mg/mL G418, 50 units/mL Pen/Strep and 1 mL heat-inactivated fetal bovine serum at 37° C. and 10% CO₂. For electrophysiological recordings, cells were plated on 35 mm dishes coated with poly-D-lysine.

Whole-cell recordings: HEK-293 cells stably expressing the PN1 sodium channel subtype were examined by whole cell voltage clamp (Hamill, et al. Pfluegers Archives 391:85-100 (1981)) using an EPC-9 amplifier and Pulse software (HEKA Electronics, Lamprecht, Germany). Experiments were performed at room temperature. Electrodes were fire-polished to resistances of 2-4 Mf-2. Voltage errors were minimized by series resistance compensation, and the capacitance transient was canceled using the EPC-9′ s built-in circuitry. Data were acquired at 50 kHz and filtered at 7-10 kHz. The bath solution consisted of 40 mM NaCl, 120 mM NMDG Cl, 1 mM KCl, 2.7 mM CaCl₂, 0.5 mM MgCl₂, 10 mM NMDG HEPES, pH 7.4, and the internal (pipet) solution contained 110 mM Cs-methanesulfonate, 5 mM NaCl, 20 mM CsCl, 10 mM CsF, 10 mM BAPTA (tetra Cs salt), 10 mM Cs HEPES, pH 7.4.

The following protocols were used to estimate the steady-state affinity of compounds for the resting and inactivated state of the channel (Kᵣ and Kᵢ, respectively):

1. 8 ms test-pulses to depolarizing voltages from −60 Mv to +50 Mv from a holding potential of −90 Mv were used to construct current-voltage relationships (IV-curves). A voltage near the peak of the IV-curve (typically −10 or 0 Mv) was used as the test-pulse voltage throughout the remainder of the experiment.

2. Steady-state inactivation (availability) curves were constructed by measuring the current activated during an 8 ms test-pulse following 10 s conditioning pulses to potentials ranging from −120 Mv to −10 Mv.

3. Compounds were applied at a holding potential at which 20-50% of the channels was inactivated and sodium channel blockage was monitored during 8 ms test pulses at intervals.

4. After the compounds equilibrated, the voltage-dependence of steady-state inactivation in the presence of compound was determined according to protocol 2) above. Compounds that block the resting state of the channel decrease the current elicited during test-pulses from all holding potentials, whereas compounds that primarily block the inactivated state shift the mid-point of the steady-state inactivation curve. The maximum current at negative holding potentials (Iₘₐₓ) and the difference in the mid-points of the steady-state inactivation curves (ΔV) in control and in the presence of a compound were used to calculate Kᵣ and Kᵢ using the following equations:

$${K}_{r} = \frac{[ Drug ] * {I}_{Max , Drug}}{{I}_{Max , Control} - {I}_{Max , Drug}}$$

$${K}_{i} = \frac{[ Drug ]}{( 1 + \frac{[ Drug ]}{{K}_{r}} ) * {e}^{\frac{- Δ V}{k}} - 1}$$

In cases where the compound did not affect the resting state, Kᵢ was calculated using the following equation:

$${K}_{i} = \frac{[ Drug ]}{{e}^{\frac{- Δ V}{k}} - 1}$$

### In Vivo Assay Using Rat CFA Model:

Unilateral inflammation was induced with a 0.2 mL injection of complete Freund's adjuvant (CFA: Mycobacterium tuberculosis, Sigma; suspended in an oil/saline (1:1) emulsion; 0.5 mg Mycobacterium/Ml) in the plantar surface of the left hindpaw. This dose of CFA produced significant hind paw swelling but the animals exhibited normal grooming behavior and weight gain over the course of the experiment. Mechanical hyperalgesia was assessed 3 days after tissue injury using a Randall-Selitto test. Repeated Measures ANOVA, followed by Dunnett's Post Hoc test.

### SNL: Mechanical Allodynia (In Vivo Assay):

Tactile allodynia was assessed with calibrated von Frey filaments using an up-down paradigm before and two weeks following nerve injury. Animals were placed in plastic cages with a wire mesh floor and allowed to acclimate for 15 min before each test session. To determine the 50% response threshold, the von Frey filaments (over a range of intensities from 0.4 to 28.8 g) were applied to the mid-plantar surface for 8 s, or until a withdrawal response occurred. Following a positive response, an incrementally weaker stimulus was tested. If there was no response to a stimulus, then an incrementally stronger stimulus was presented. After the initial threshold crossing, this procedure was repeated for four stimulus presentations per animal per test session. Mechanical sensitivity was assessed 1 and 2 hr post oral administration of the test compound.

The compounds described in this invention displayed sodium channel blocking activity of from about <0.1 μM to about <50 μM in the in vitro assays described above. It is advantageous that the compounds display sodium channel blocking activity of <5 μM in the in vitro assays. It is more advantageous that the compounds display sodium channel blocking activity of <1 μM in the in vitro assays. It is even more advantageous that the compounds display sodium channel blocking activity of <0.5 μM in the in vitro assays. It is still more advantageous that the compounds display sodium channel blocking activity of <0.1 μM in the in vitro assays.

### Mouse Epilepsy Model: Maximal Electroconvulsive Seizures (MES) (In Vivo Assay):

The threshold for maximal (tonic hind limb extension and clonic paddling of hind limbs) electroshock seizures in male C57BL6 mice was determined using auricular electrodes connected to a Basile Electroconvulsive Device (57800 Basile) designed for inducing convulsions in research animals. For anticonvulsant testing, the following parameters were utilized: Frequency=100 Hz; Pulse width=0.7 mSec; Shock duration=0.5 Sec; Current=18 mA. In untreated or vehicle treated mice, these parameters produced typical seizures consisting of tonic flexor, tonic extensor and clonic phases, without any mortality. The percentage of animals having clonic seizure and the percentage of animals having tonic seizure were recorded. Compounds with anticonvulsant activity protected against tonic and clonic seizures.

What is claimed is:

1. A compound represented by formula (I): 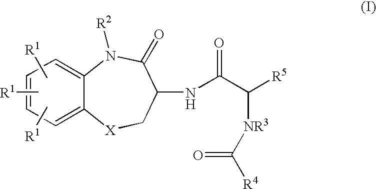
and pharmaceutically acceptable salts thereof, wherein
each R¹ is independently selected from the group consisting of hydrogen,
halogen,
cyano,
C₁₋₆ alkyl, unsubstituted or substituted with one to five halogens, and
C₁₋₆ alkoxy, unsubstituted or substituted with one to five halogens;
R² is independently selected from the group consisting of
hydrogen,
C₁₋₆ alkyl, unsubstituted or substituted with one to six substituents selected from halogen and hydroxy,
C₁₋₆ alkenyl,
C₁₋₆ alkynyl,
C₁₋₆ alkoxy-C₁₋₆ alkylene, unsubstituted or substituted with one to six halogens,
C₁₋₆ cycloalkyl, wherein cycloalkyl is unsubstituted or substituted with one to six substituents independently selected from halogen, cyano, C₁₋₆ alkyl and
C₁₋₆ alkoxy, wherein alkyl and alkoxyl are unsubstituted or substituted with one to six halogens, and
C₁₋₆ cycloalkyl-C₁₋₆alkylene, wherein cycloalkyl is unsubstituted or substituted with one to six substituents independently selected from halogen, cyano, C₁₋₆ alkyl and C₁₋₆ alkoxy, wherein alkyl and alkoxyl are unsubstituted or substituted with one to six halogens;
R³ is independently selected from the group consisting of
hydrogen and
C₁₋₆ alkyl;
R⁴ is independently selected from the group consisting of
C₁₋₁₀ alkyl, unsubstituted or substituted with one to six halogens,
C₁₋₁₀ alkoxy, unsubstituted or substituted with one to six halogens,
C₁₋₁₀ cycloalkyl-C₀₋₆ alkylene, wherein cycloalkyl is unsubstituted or substituted with one to six substituents independently selected from halogen, cyano, C₁₋₆ alkyl and C₁₋₆ alkoxy, wherein alkyl and alkoxyl are unsubstituted or substituted with one to six halogens,
—(CH₂)ₘ-aryl wherein m is 0, 1, 2 or 3, and wherein aryl is unsubstituted or substituted with one to five substituents independently selected from halogen, cyano, C₁₋₆ alkyl and C₁₋₆ alkoxy, wherein alkyl and alkoxy are unsubstituted or substituted with one to six halogens, and
—(CH₂)ₘ-heteroaryl wherein m is 0, 1, 2 or 3, and wherein heteroaryl is unsubstituted or substituted with one to five substituents independently selected from halogen, C₁₋₆ alkyl and C₁₋₆ alkoxy, wherein alkyl and alkoxy are unsubstituted or substituted with one to six halogens;
R⁵ is independently selected from the group consisting of
—(CH₂)ₙ-aryl wherein n is 0, 1, or 2, and wherein aryl is unsubstituted or substituted with one to five substituents independently selected from hydroxy, halogen, cyano, C₁₋₆ alkyl and C₁₋₆ alkoxy, wherein alkyl and alkoxy are unsubstituted or substituted with one to six halogens,
—(CH₂)ₙ-heteroaryl wherein n is 0, 1 or 2, and wherein aryl is unsubstituted or substituted with one to five substituents independently selected from halogen, C₁₋₆ alkyl and C₁₋₆ alkoxy, wherein alkyl and alkoxy are unsubstituted or substituted with one to six halogens;
X is independently selected from the group consisting of
oxygen,
nitrogen, unsubstituted or substituted with one R⁶ as defined herein,
sulfur,
sulfoxide, and
sulfone;
R⁶ is independently selected from the group consisting of
C₁₋₁₀ alkyl, unsubstituted or substituted with one to six halogens,
C₁₋₁₀ alkoxy, unsubstituted or substituted with one to six halogens,
C₁₋₁₀ cycloalkyl-C₀₋₆ alkylene, wherein cycloalkyl is unsubstituted or substituted with one to six substituents independently selected from halogen, cyano, C₁₋₆ alkyl and C₁₋₆ alkoxy, wherein alkyl and alkoxyl are unsubstituted or substituted with one to six halogens,
—(CH₂)ₚ-aryl wherein p is 0, 1, 2 or 3, and wherein aryl is unsubstituted or substituted with one to five substituents independently selected from halogen, cyano, C₁₋₆ alkyl and C₁₋₆ alkoxy, wherein alkyl and alkoxy are unsubstituted or substituted with one to six halogens, and
—(CH₂)ₚ-heteroaryl wherein p is 0, 1, 2 or 3, and wherein heteroaryl is unsubstituted or substituted with one to five substituents independently selected from halogen, C₁₋₆ alkyl and C₁₋₆ alkoxy, wherein alkyl and alkoxy are unsubstituted or substituted with one to six halogens.

2. The compound according to claim 1 represented by formula (Ia) 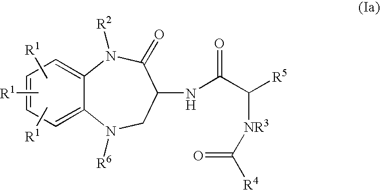
and pharmaceutically acceptable salts thereof, wherein R¹, R², R³, R⁴, R⁵ and R⁶ are as previously defined.

3. A compound according to claim 2 represented by formula (Ib) 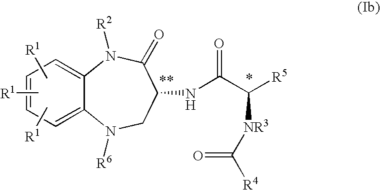
and pharmaceutically acceptable salts thereof, wherein the carbon atoms marked with * and ** have the stereochemical configurations depicted in formula (Ib) and R¹, R², R³, R⁴, R⁵ and R⁶ are as previously defined.

4. The compound according to claim 3 represented by formula (Ic) 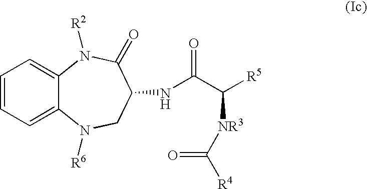
and pharmaceutically acceptable salts thereof, wherein R², R³, R⁴, R⁵ and R⁶ are as previously defined.

5. The compound according to claim 4, wherein:
R² is independently selected from the group consisting of
hydrogen,
C₁₋₆ alkyl, unsubstituted or substituted with one to six substituents selected from halogen and hydroxy,
C₁₋₆ alkenyl, and
C₁₋₆ alkoxy-C₁₋₆ alkylene, unsubstituted or substituted with one to six halogens;
R³ is hydrogen;
R⁴ is independently selected from the group consisting of
C₁₋₆ alkyl, unsubstituted or substituted with one to six halogens,
C₁₋₆ alkoxy, unsubstituted or substituted with one to six halogens,
C₃₋₆ cycloalkyl-C₀₋₆ alkylene, wherein cycloalkyl is unsubstituted or substituted with one to six substituents independently selected from halogen, cyano, C₁₋₆ alkyl and C₁₋₆ alkoxy, wherein alkyl and alkoxy are unsubstituted or substituted with one to six halogens, and
phenyl, wherein phenyl is unsubstituted or substituted with one to five substituents independently selected from halogen, cyano, C₁₋₆ alkyl and C₁₋₆ alkoxy, wherein alkyl and alkoxy are unsubstituted or substituted with one to six halogens,
R⁵ is
—CH₂-phenyl, wherein phenyl is unsubstituted or substituted with one to five substituents independently selected from hydroxy, halogen, cyano, C₁₋₆ alkyl and C₁₋₆ alkoxy, wherein alkyl and alkoxy are unsubstituted or substituted with one to six halogens; and
R⁶ is independently selected from the group consisting of
C₁₋₁₀ alkyl, unsubstituted or substituted with one to six halogens,
C₁₋₁₀ cycloalkyl-C₀₋₆ alkylene, wherein cycloalkyl is unsubstituted or substituted with one to six substituents independently selected from halogen, cyano, C₁₋₆ alkyl and C₁₋₆ alkoxy, wherein alkyl and alkoxyl are unsubstituted or substituted with one to six halogens, and
—(CH₂)ₚ-phenyl wherein p is 0, 1, 2 or 3, and wherein phenyl is unsubstituted or substituted with one to five substituents independently selected from halogen, cyano, C₁₋₆ alkyl and C₁₋₆ alkoxy, wherein alkyl and alkoxy are unsubstituted or substituted with one to six halogens.

6. The compound according to claim 1 represented by formula (Id) 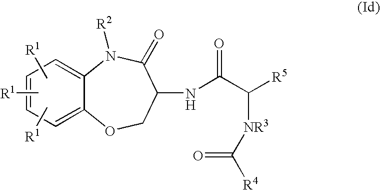
and pharmaceutically acceptable salts thereof, wherein R¹, R², R³, R⁴ and R⁵ are as previously defined.

7. The compound according to claim 6 represented by formula (Ie) 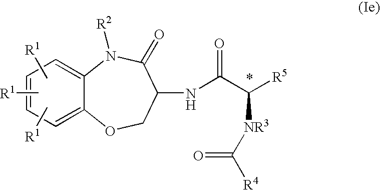
and pharmaceutically acceptable salts thereof, wherein the carbon atom marked with a * has the stereochemical configuration depicted in formula (Ie) and R¹, R², R³, R⁴ and R⁵ are as previously defined.

8. The compound according to claim 7, wherein:
R² is independently selected from the group consisting of
hydrogen,
C₁₋₆ alkyl, unsubstituted or substituted with one to six substituents selected from halogen and hydroxy,
C₁₋₆ alkenyl, and
C₁₋₆ alkoxy-C₁₋₆ alkylene, unsubstituted or substituted with one to six halogens;
R³ is hydrogen;
R⁴ is independently selected from the group consisting of
C₁₋₆ alkyl, unsubstituted or substituted with one to six halogens,
C₁₋₆ alkoxy, unsubstituted or substituted with one to six halogens,
C₃₋₆ cycloalkyl-C₁₋₆ alkylene, wherein cycloalkyl is unsubstituted or substituted with one to six substituents independently selected from halogen, cyano, C₁₋₆ alkyl and C₁₋₆ alkoxy, wherein alkyl and alkoxy are unsubstituted or substituted with one to six halogens, and
phenyl, wherein phenyl is unsubstituted or substituted with one to five substituents independently selected from halogen, cyano, C₁₋₆ alkyl and C_{1 -6} alkoxy, wherein alkyl and alkoxy are unsubstituted or substituted with one to six halogens; and
R⁵ is
—CH₂-phenyl, wherein phenyl is unsubstituted or substituted with one to five substituents independently selected from hydroxy, halogen, cyano, C₁₋₆ alkyl and C₁₋₆ alkoxy, wherein alkyl and alkoxy are unsubstituted or substituted with one to six halogens.

9. The compound according to claim 1 represented by formula (If) 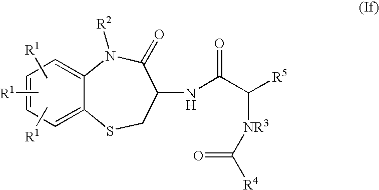
and pharmaceutically acceptable salts thereof, wherein R¹, R², R⁴ and R⁵ are as previously defined.

10. The compound according to claim 9 represented by formula (Ig) 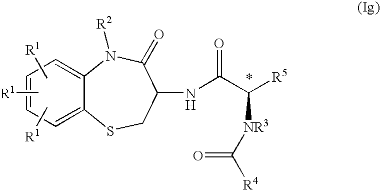
and pharmaceutically acceptable salts thereof, wherein the carbon atom marked with an * has the stereochemical configuration as depicted in formula (Ig) and R¹, R², R³, R⁴ and R⁵ are as previously defined.

11. The compound according to claim 10, wherein:
R² is independently selected from the group consisting of
hydrogen,
C₁₋₆ alkyl, unsubstituted or substituted with one to six substituents selected from halogen and hydroxy,
C₁₋₆ alkenyl, and
C₁₋₆ alkoxy-C₁₋₆ alkylene, unsubstituted or substituted with one to six halogens;
R³ is hydrogen;
R⁴ is independently selected from the group consisting of
C₁₋₆ alkyl, unsubstituted or substituted with one to six halogens,
C₁₋₆ alkoxy, unsubstituted or substituted with one to six halogens,
C₃₋₆ cycloalkyl-C₀₋₆ alkylene, wherein cycloalkyl is unsubstituted or substituted with one to six substituents independently selected from halogen, cyano, C₁₋₆ alkyl and C₁₋₆ alkoxy, wherein alkyl and alkoxy are unsubstituted or substituted with one to six halogens, and
phenyl, wherein phenyl is unsubstituted or substituted with one to five substituents independently selected from halogen, cyano, C₁₋₆ alkyl and C₁₋₆ alkoxy, wherein alkyl and alkoxy are unsubstituted or substituted with one to six halogens; and
R⁵ is
—CH₂-phenyl, wherein phenyl is unsubstituted or substituted with one to five substituents independently selected from hydroxy, halogen, cyano, C₁₋₆ alkyl and C₁₋₆ alkoxy, wherein alkyl and alkoxy are unsubstituted or substituted with one to six halogens.

12. The compound according to claim 1 selected from the following table:

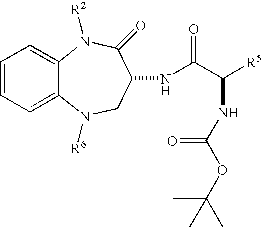

| R² | R⁵ | R⁶ |
|---|---|---|
| 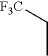 | 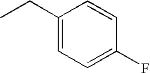 | 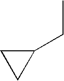 |
| H | 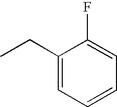 | H |
| Me | 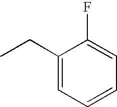 | H |
| Me | 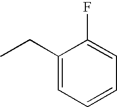 | Me |
| Me | 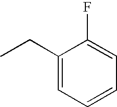 | 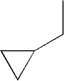 |
| Me | 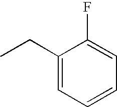 | 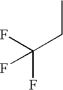 |
| Me | 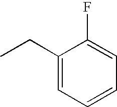 | 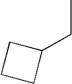 |
| Me | 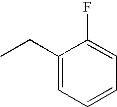 | 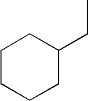 |
| Me | 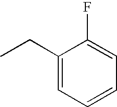 | 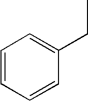 |
| i-Pr | 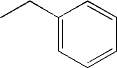 | H |
| i-Pr | 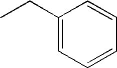 | 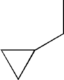 |
| i-Pr | 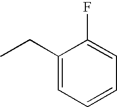 | 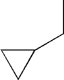 |
| i-Pr | 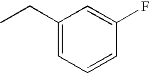 | 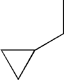 |
| i-Pr | 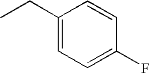 | 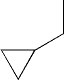 |
| i-Pr | 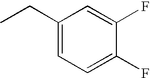 | 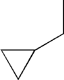 |
| i-Pr | 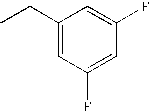 | 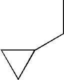 |
| i-Pr | 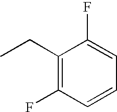 | 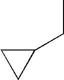 |
| i-Pr | 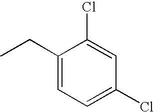 | 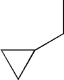 |
| i-Pr | 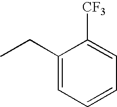 | 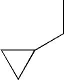 |
| i-Pr | 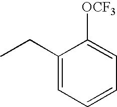 | 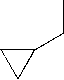 |
| i-Pr | 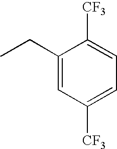 | 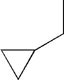 |
| 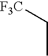 | 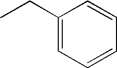 | 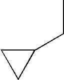 |
| 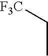 | 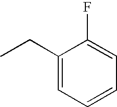 | 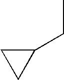 |
| 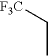 | 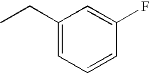 | 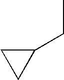 |
| 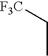 | 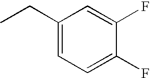 | 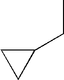 |
| 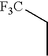 | 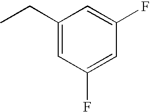 | 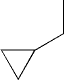 |
| 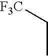 | 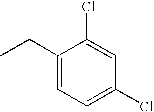 | 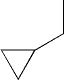 |
| 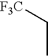 | 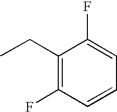 | 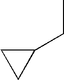 |
| 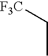 | 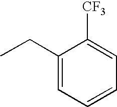 | 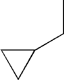 |
| 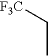 | 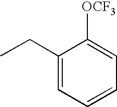 | 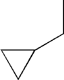 |
| 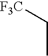 | 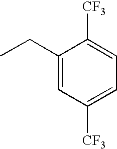 | 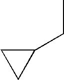 |

and pharmaceutically acceptable salts of any of the foregoing compounds.

13. The compound according to claim 1 selected from the following table:

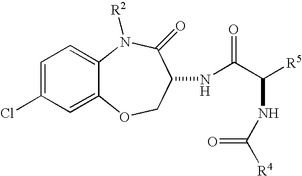

| R² | R⁴ | R⁵ |
|---|---|---|
| i-Pr | 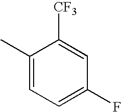 | 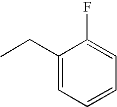 |
| 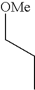 | OC(CH₃)₃ | 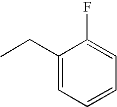 |
| 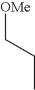 | OC(CH₃)₃ | 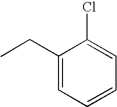 |
| 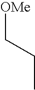 | 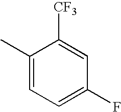 | 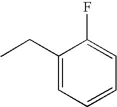 |
| 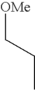 | 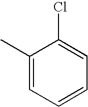 | 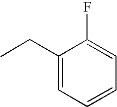 |
| 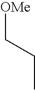 | 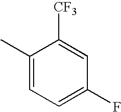 | 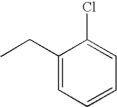 |
| 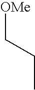 | 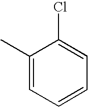 | 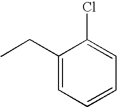 |
| i-Pr | OC(CH₃)₃ | 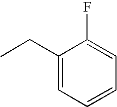 |
| i-Pr | OC(CH₃)₃ | 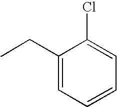 |
| i-Pr | OC(CH₃)₃ | 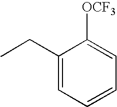 |
| i-Pr | 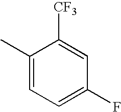 | 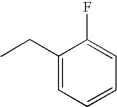 |
| i-Pr | OC(CH₃)₃ | 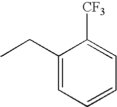 |
| i-Pr | 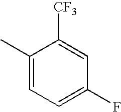 | 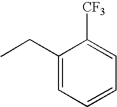 |
| 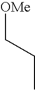 | OC(CH₃)₃ | 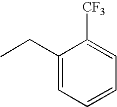 |
| 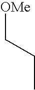 | OC(CH₃)₃ | 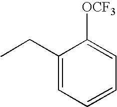 |
| 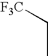 | OC(CH₃)₃ | 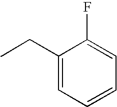 |
| 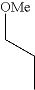 | 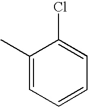 | 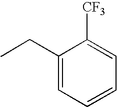 |
| 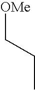 | 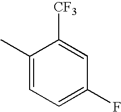 | 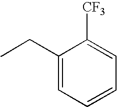 |
| 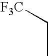 | 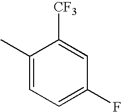 | 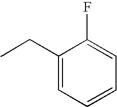 |
| 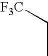 | 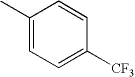 | 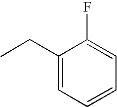 |
| i-Pr | 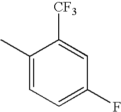 | 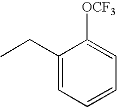 |
| 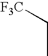 | 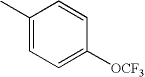 | 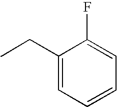 |
| 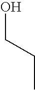 | OC(CH₃)₃ | 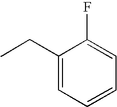 |
| 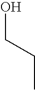 | OC(CH₃)₃ | 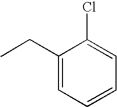 |
| i-Pr | 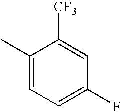 | 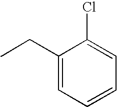 |
| 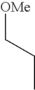 | 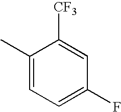 | 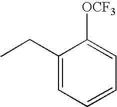 |
| 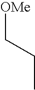 | 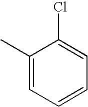 | 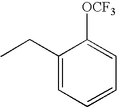 |
| 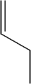 | OC(CH₃)₃ | 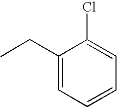 |
| 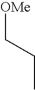 | OC(CH₃)₃ | 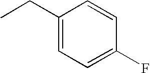 |
| 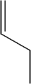 | 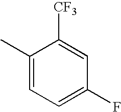 | 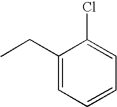 |
| 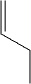 | 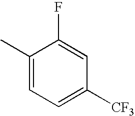 | 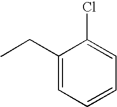 |
| 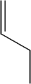 | 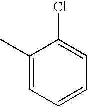 | 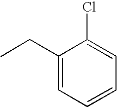 |
| 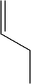 | 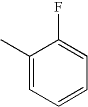 | 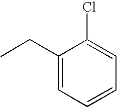 |
| 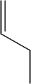 | 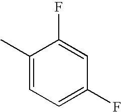 | 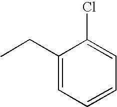 |
| i-Pr | OC(CH₃)₃ | 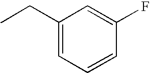 |
| i-Pr | 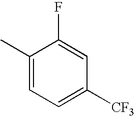 | 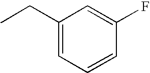 |
| 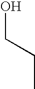 | OC(CH₃)₃ | 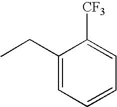 |
| i-Pr | 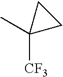 | 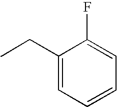 |
| i-Pr | 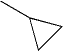 | 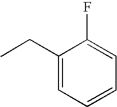 |
| i-Pr | 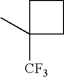 | 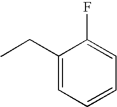 |
| i-Pr | 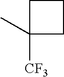 | 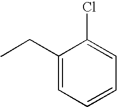 |
| i-Pr | 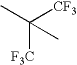 | 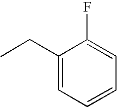 |
| i-Pr | 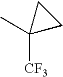 | 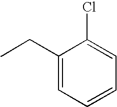 |
| i-Pr | 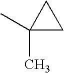 | 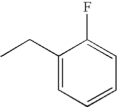 |
| i-Pr | 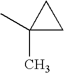 | 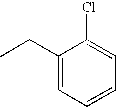 |
| i-Pr | 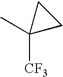 | 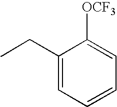 |
| i-Pr | 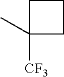 | 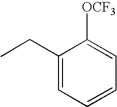 |
| i-Pr | 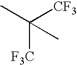 | 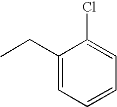 |
| i-Pr | 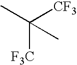 | 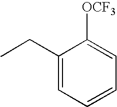 |
| i-Pr | 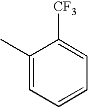 | 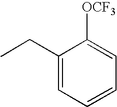 |
| i-Pr | 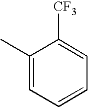 | 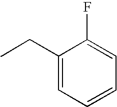 |
| i-Pr | 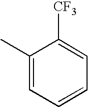 | 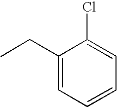 |
| i-Pr | t-Bu | 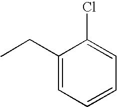 |
| i-Pr | t-Bu | 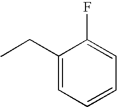 |
| i-Pr | 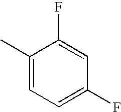 | 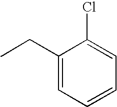 |
| i-Pr | 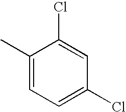 | 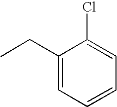 |
| i-Pr | 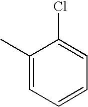 | 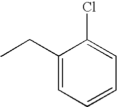 |
| i-Pr | 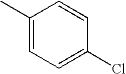 | 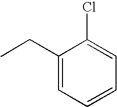 |

and pharmaceutically acceptable salts of any of the foregoing compounds.

14. The compound according to claim 1 selected from the following able:

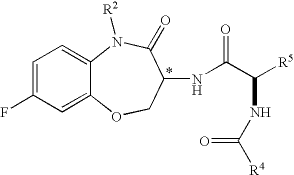

| * | R² | R⁴ | R⁵ |
|---|---|---|---|
| R | i-Pr | 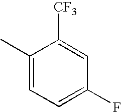 | 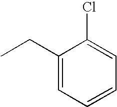 |
| R | i-Pr | OC(CH₃)₃ | 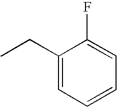 |
| S | i-Pr | OC(CH₃)₃ | 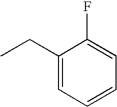 |
| R | i-Pr | 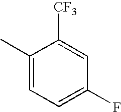 | 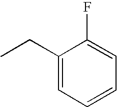 |
| R | i-Pr | 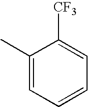 | 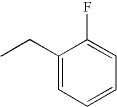 |
| S | i-Pr | 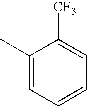 | 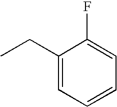 |
| R | i-Pr | 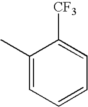 | 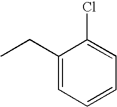 |
| S | i-Pr | 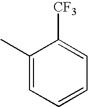 | 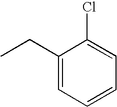 |
| R | i-Pr | OC(CH₃)₃ | 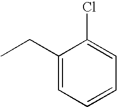 |
| S | i-Pr | OC(CH₃)₃ | 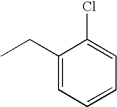 |
| R | i-Pr | 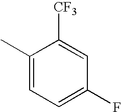 | 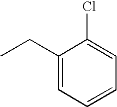 |
| S | i-Pr | 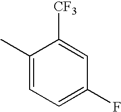 | 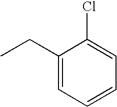 |
| R | i-Pr | 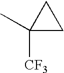 | 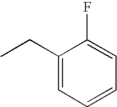 |
| R | i-Pr | t-Bu | 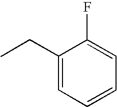 |
| S | i-Pr | t-Bu | 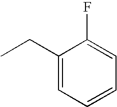 |
| R | i-Pr | OC(CH₃)₃ | 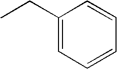 |
| S | i-Pr | OC(CH₃)₃ | 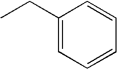 |
| R | i-Pr | 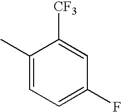 | 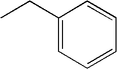 |
| R | i-Pr | 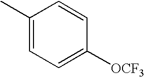 | 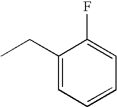 |
| R | i-Pr | 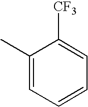 | 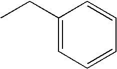 |
| S | i-Pr | 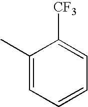 | 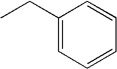 |
| R | i-Pr | 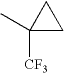 | 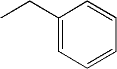 |
| R | i-Pr | 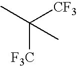 | 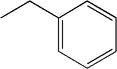 |
| R | i-Pr | 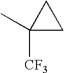 | 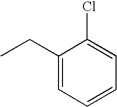 |
| S | i-Pr | 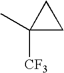 | 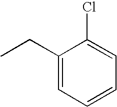 |

and pharmaceutically acceptable salts of any of the foregoing compounds.

15. The compound according to claim 1 selected from the following table:

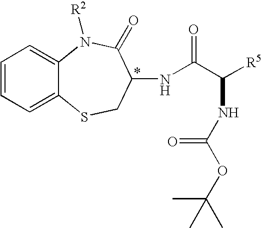

| * | R² | R⁵ |
|---|---|---|
| R | i-Pr | 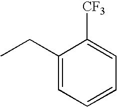 |
| R | H | 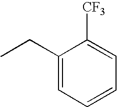 |
| R | 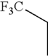 | 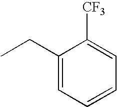 |
| R | 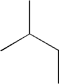 | 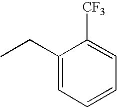 |
| S | H | 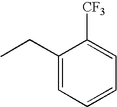 |
| S | 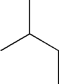 | 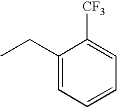 |
| S | i-Pr | 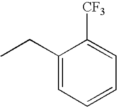 |

and pharmaceutically acceptable salts of any of the foregoing compounds.

16. The compound according to claim 1 selected from the following: 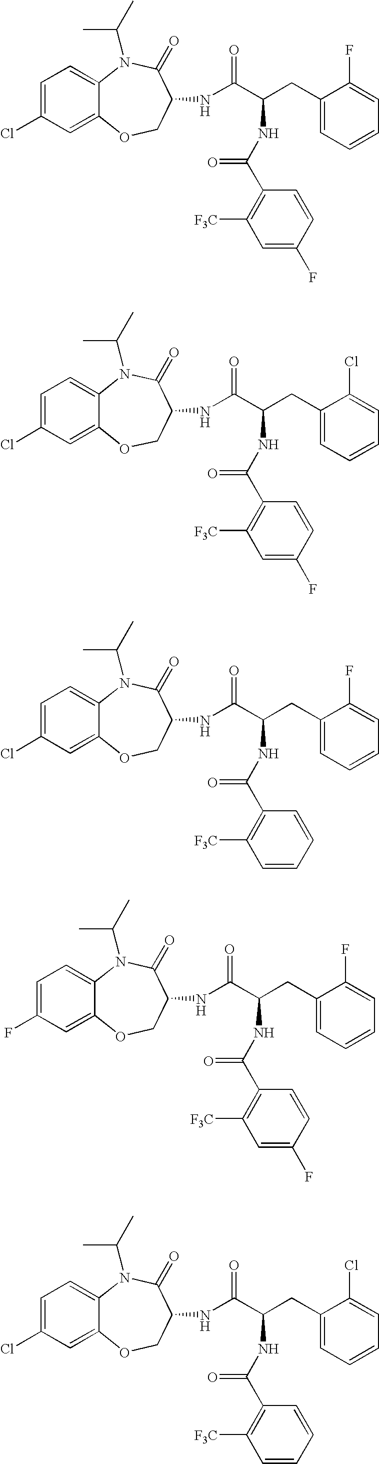 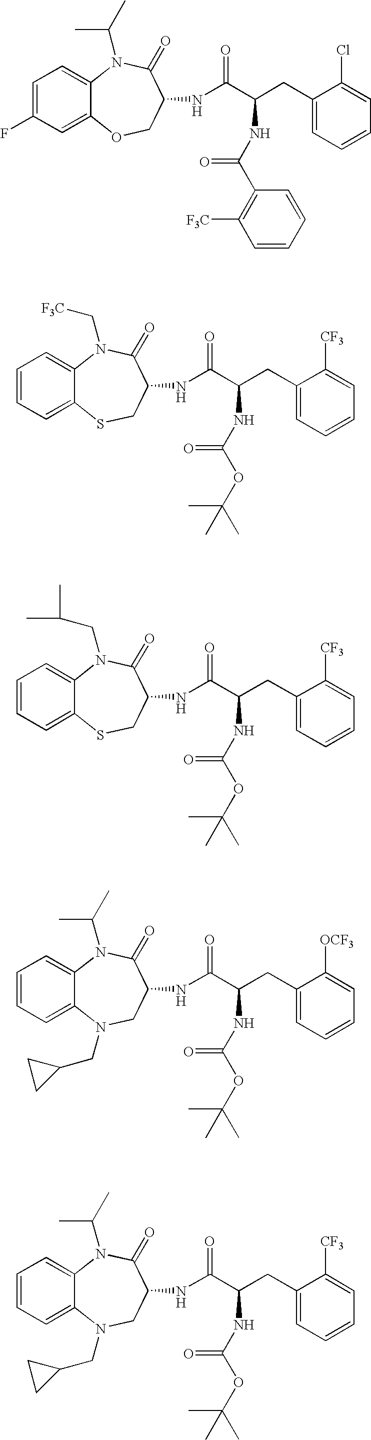 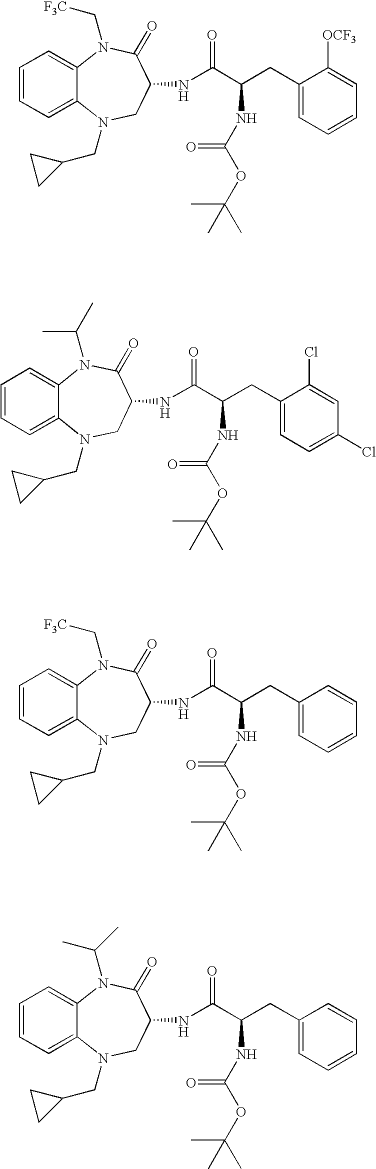
and pharmaceutically acceptable salts of any of the foregoing compounds.

17. A pharmaceutical composition comprising a therapeutically effective amount of a compound according to claim 1, or a pharmaceutically acceptable salt thereof, and a pharmaceutically acceptable carrier.

18. The pharmaceutical composition according to claim 17, further comprising a second therapeutic agent selected from the group consisting of: i) opiate agonists, ii) opiate antagonists, iii) calcium channel antagonists, iv) 5HT receptor agonists, v) 5HT receptor antagonists vi) sodium channel antagonists, vii) NMDA receptor agonists, viii) NMDA receptor antagonists, ix) COX-2 selective inhibitors, x) NK1 antagonists, xi) non-steroidal anti-inflammatory drugs, xii) selective serotonin reuptake inhibitors, xiii) selective serotonin and norepinephrine reuptake inhibitors, xiv) tricyclic antidepressant drugs, xv) norepinephrine modulators, xvi) lithium, xvii) valproate, xviii) neurontin, and xix) pregabalin.

19. A method of treatment or prevention of pain comprising the step of administering to a patient in need thereof a therapeutically effective amount, or a prophylactically effective amount, of a compound according to claim 1, or a pharmaceutically acceptable salt thereof.

20. A method of treatment or prevention of one or more of the following condition in a patient in need thereof:
(1) chronic, visceral, inflammatory and/or neuropathic pain syndromes;
(2) pain resulting from, or associated with, traumatic nerve injury, nerve compression or entrapment, postherpetic neuralgia, trigeminal neuralgia, diabetic neuropathy, cancer and/or chemotherapy,
(3) chronic lower back pain;
(4) phantom limb pain; and
(5) HIV- and HIV treatment-induced neuropathy, chronic pelvic pain, neuroma pain, complex regional pain syndrome, chronic arthritic pain and related neuralgias;
comprising the step of administering to a patient in need thereof a therapeutically effective amount, or a prophylactically effective amount, of a compound according to claim 1, or a pharmaceutically acceptable salt thereof.
